(19) United States
(12) Patent Application Publication
(10) Pub. No.: US 2025/0288213 A1
(43) Pub. Date: Sep. 18, 2025

(54) DEVICES AND METHODS FOR NON-INVASIVE MEASUREMENT OF INTRAVASCULAR PRESSURE

(71) Applicant: 1000735308 Ontario Inc., Toronto (CA)

(72) Inventors: Ankit Garg, Toronto (CA); Matthew Vargas, Encinitas, CA (US)

(21) Appl. No.: 19/081,613

(22) Filed: Mar. 17, 2025

Related U.S. Application Data
(60) Provisional application No. 63/566,379, filed on Mar. 17, 2024.

(51) Int. Cl. A61B 5/021; A61B 5/00

(52) U.S. Cl. CPC A61B 5/021; A61B 5/6823; A61B 5/742; A61B 2562/0219; A61B 2562/0223

(57) ABSTRACT

Devices for measuring blood pressure are disclosed herein. According to some embodiments, the present technology includes a device comprising a housing configured to be secured to a patient's body, a pressure sensor, a position sensor, at least one processor in communication with the pressure sensor and the position sensor, and memory. Program instructions stored on the memory, when executed by the at least one processor, cause the device to perform functions comprising determining a blood pressure of the patient based on the pressure data and the position data and outputting the blood pressure via the output device.

### CROSS-REFERENCE TO RELATED APPLICATION(S)

The present application claims the benefit of priority to U.S. Provisional Application No. 63/566,379, filed Mar. 17, 2024, which is incorporated by reference herein in its entirety.

### TECHNICAL FIELD

The present technology relates to devices and methods for non-invasive measurement of one or more physiological parameters of a patient, and more particularly, to devices and methods for non-invasive measurement of intravascular pressure.

### BACKGROUND

Acute heart failure is the cause of over one million hospitalizations per year. Fluid overload drives heart failure hospitalizations, and intravenous diuretics are the current standard of treatment. Despite receipt of intravenous diuretics, heart failure patients have a 30% readmission rate, at least in part due to patients being released under the mistaken assessment that complete decongestion has been achieved. For example, heart failure patients are two times more likely to be readmitted if residual congestion is present at discharge, and studies have shown that 33% of heart failure patients are discharged with residual congestion. Errors in determining whether a patient remains congested can also affect the patient's length of stay (and thus cost of treatment). Intravenous diuretics are prematurely terminated in one in four heart failure patients, for instance, requiring re-initiation of intravenous therapy and extending the patient's stay by three days on average.

Hemodynamic monitoring of central venous pressure is considered the gold standard in managing congestion. Invasive measurements provide accurate results but require insertion of a pressure monitoring probe directly into the patient's subclavian or internal jugular vein in order to measure central venous pressure, or directly into the patient's right heart to measure pulmonary pressure. A widely used non-invasive technique includes the use of an off-the-shelf pressure transducer connected in line with the patient's intravenous diuretic bag and wired to a separate cardiac monitor that outputs central venous pressure waveforms on a display. However, the equipment and personnel required for such invasive and non-invasive measurements only exist in the intensive care unit or operating rooms, and 90% of heart failure patients receive care on general medical floors. Instead, venous pressure is assessed via a crude physical exam or bedside ultrasound, both of which are inaccurate, and the latter requires a physician and access to an ultrasound machine.

Accordingly, there exists a need for improved methods for monitoring venous pressure in non-critical (e.g., general ward) settings.

### SUMMARY

The present technology is directed to monitoring devices configured to consistently provide accurate venous pressure measurements (including peripheral venous pressure, or PVP) in non-critical care settings (ward) by non-advanced care providers, for example to assess decongestion in patients with heart failure. The subject technology is illustrated, for example, according to various aspects described below, including with reference to FIGS. 1-12. Various examples of aspects of the subject technology are described as numbered examples (1, 2, 3, etc.) for convenience. These are provided as examples and do not limit the subject technology.

1. A non-invasive device for measuring a patient's blood pressure, the device comprising:
a housing configured to be worn on a body of the patient,
a first port carried by the housing, the first port configured to be detachably coupled to an outlet end of a fluid source;
a second port carried by the housing, the second port configured to be detachably coupled to a proximal end of a catheter;
a fluid channel extending from the first port through the housing to the second port such that the first and second ports are fluidly coupled via the fluid channel and such that, when the first port is coupled to the fluid source and a distal end of the catheter is disposed in a patient's blood vessel, a fluid from the fluid source flows through the fluid channel and into the blood vessel;
a pressure sensor carried by the housing, wherein the pressure sensor is operatively coupled to the fluid channel and configured to measure a fluid pressure along the channel;
a position sensor carried by the housing; and
a controller coupled to the pressure sensor and the position sensor, wherein the controller comprises memory and processing circuitry configured to determine a blood pressure of the patient via data received from the pressure sensor and/or the position sensor.

2. The device of Example 1, wherein the position sensor is configured to measure the position of the housing with three degrees of freedom.

3. The device of Example 1, wherein the position sensor is configured to measure the position of the housing with six degrees of freedom.

4. The device of Example 1, wherein the position sensor is configured to measure the position of the housing with nine degrees of freedom.

5. The device of any one of Examples 1 to 4, wherein the position sensor comprises an accelerometer.

6. The device of any one of Examples 1 to 5, wherein the position sensor comprises a gyroscope.

7. The device of any one of Examples 1 to 6, wherein the position sensor comprises a magnetometer.

8. The device of Example 1, wherein the position sensor comprises an inertial measurement unit.

9. The device of any one of Examples 1 to 8, further comprising a display carried by the housing, and wherein the controller is coupled to display and configured to output the determined blood pressure on the display.

10. The device of Example 9, wherein the determined blood pressure is output as a number.

11. The device of Example 9 or Example 10, wherein the determined blood pressure is not displayed as a waveform.

12. The device of any one of Examples 9 to 11, wherein the controller is configured to output instructions for a user on the display.

13. The device of any one of Examples 1 to 12, wherein the blood pressure comprises mean venous pressure.

14. The device of any one of Examples 1 to 13, wherein the blood pressure comprises peripheral venous pressure (“PVP”).

15. The device of any one of Examples 1 to 14, wherein the blood pressure comprises central venous pressure (“CVP”).

16. The device of any one of Examples 1 to 15, wherein the controller is configured to determine a PVP, and based on the PVP, determine a CVP.

17. The device of Example 16, further comprising a display carried by the housing, and wherein the controller is configured to output both the determined PVP and the determined CVP on the display.

18. The device of any one of Examples 1 to 17, further comprising a third sensor carried by the housing, wherein the third sensor comprises at least one of an antenna sensor, a capacitive sensor, a resistive sensor, a bioimpedance sensor, a piezoelectric sensor, an ECG sensor, and/or an optical sensor.

19. The device of any one of Examples 1 to 18, further comprising a power source.

20. The device of any one of Examples 1 to 19, further comprising a wireless transmitter.

21. The device of any one of Examples 1 to 20, wherein the controller is disposed within the housing.

22. The device of any one of Examples 1 to 20, wherein the controller is a separate device that is wirelessly coupled to the device.

23. A device for measuring a patient's blood pressure, the device comprising:
a pressure monitoring device comprising:
   a housing configured to be worn on a body of the patient,
   a first port carried by the housing, the first port configured to be detachably coupled to an outlet end of a fluid source;
   a second port carried by the housing, the second port configured to be detachably coupled to a proximal end of a catheter;
   a fluid channel extending from the first port through the housing to the second port such that the first and second ports are fluidly coupled via the fluid channel and such that, when the first port is coupled to the fluid source and a distal end of the catheter is disposed in a patient's blood vessel, a fluid from the fluid source flows through the fluid channel and into the blood vessel;
   a pressure sensor carried by the housing, wherein the pressure sensor is operatively coupled to the fluid channel and configured to measure a fluid pressure along the channel;
   a position sensor carried by the housing; and
   a controller coupled to the pressure sensor and the position sensor, wherein the controller comprises memory and processing circuitry configured to determine a blood pressure of the patient via data received from the pressure sensor and/or the position sensor; and
a dock configured to be secured to the patient's skin, wherein the dock is configured to be detachably coupled to the device thereby securing the device to the patient.

24. The system of Example 23, wherein the dock includes an adhesive configured to be temporarily adhered to the patient's skin.

25. The system of Example 23 or Example 24, wherein the device is configured to sense secure attachment to the dock.

26 The system of Example 25, wherein the device is configured to communicate to a user when the device is secured received by the dock.

27. A method, comprising:
securing a device to a body of a patient, the device including a position sensor and a pressure sensor and configured to measure an intravascular pressure of the patient;
coupling the device to a cannula configured to be inserted into the patient's blood vessel and a fluid source containing a fluid for intravenous delivery to the patient, thereby enabling fluid flow from the fluid source through the device and into the blood vessel;
obtaining pressure data via the pressure sensor;
obtaining position data characterizing a position of the device via the position sensor; and
viewing a blood pressure value on a display of the device, the blood pressure value based on the pressure data and the position data.

28. A method, comprising:
securing a device to a body of a patient, the device including a position sensor and a pressure sensor and configured to measure an intravascular pressure of the patient;
coupling the device to a cannula configured to be inserted into the patient's blood vessel;
obtaining pressure data via the pressure sensor; obtaining position data characterizing a position of the device via the position sensor; and
viewing a blood pressure value on a display of the device, the blood pressure value based on the pressure data and the position data.

29. The method of Example 27 or Example 28, wherein the device includes a display, and wherein the method further comprises instructing the user to perform a zeroing procedure via the display.

30. The method of any one of Examples 27 to 29, wherein the device includes a display, and wherein the method further comprises alerting the user that the patient has moved via the display.

31. The method of any one of Examples 27 to 30, wherein the device includes a display, and wherein the method further comprises instructing the user to reposition the patient and/or device via the display.

32. The method of any one of Examples 27 to 30, wherein the device includes a display, and wherein the method further comprises instructing the user to perform a patency check via the display.

33. The method of any one of Examples 27 to 31, wherein the device includes a display, and wherein the method further comprises alerting the user that the fluid channel is obstructed.

34. The method of any one of Examples 27 to 33, wherein the device includes a display, and wherein the method further comprises instructing the user to take a blood pressure measurement via the display.

35. The method of any one of Examples 27 to 34, wherein securing the device to the body of the patient comprises securing a docking structure to the body of the patient and securing the pressure monitoring device to the docking structure.

36. The method of Example 35, further comprising alerting the user, via an output device on the device, that the device is secured to the docking structure.

37. A device for measuring intravascular pressure, the device comprising:
a housing configured to be secured to a patient's body;
an output device carried by the housing;
a pressure sensor configured to obtain pressure data;
a position sensor configured to obtain position data;
at least one processor in communication with the pressure sensor and the position sensor;
at least one tangible, non-transitory computer-readable medium; and
program instructions stored on the at least one tangible, non-transitory computer-readable medium that, when executed by the at least one processor, cause the device to perform functions comprising:
   based on position data received from the position sensor, indicate to a user that the device and/or patient is in position for a pressure measurement or that the device and/or patient should be repositioned, and
   based on pressure data received from the pressure sensor, determine a blood pressure of the patient and cause the output device to indicate to the user the blood pressure of the patient.

38. The device of Example 37, wherein the position sensor is configured to measure the position of the housing with three degrees of freedom.

39. The device of Example 37, wherein the position sensor is configured to measure the position of the housing with six degrees of freedom.

40. The device of Example 37, wherein the position sensor is configured to measure the position of the housing with nine degrees of freedom.

41. The device of any one of Examples 37 to 40, wherein the position sensor comprises an accelerometer.

42. The device of any one of Examples 37 to 41, wherein the position sensor comprises a gyroscope.

43. The device of any one of Examples 37 to 42, wherein the position sensor comprises a magnetometer.

44. The device of Example 37, wherein the position sensor comprises an inertial measurement unit.

45. The device of any one of Examples 37 to 44, wherein the output device includes a display carried by the housing, and wherein the processor is coupled to display and configured to output the determined blood pressure on the display.

46. The device of Example 45, wherein the determined blood pressure is output as a number.

47. The device of Example 45 or Example 46, wherein the determined blood pressure is not displayed as a waveform.

48. The device of any one of Examples 45 to 47, wherein the processor is configured to output instructions for a user on the display.

49. The device of any one of Examples 37 to 48, wherein the blood pressure comprises mean venous pressure.

50. The device of any one of Examples 37 to 49, wherein the blood pressure comprises peripheral venous pressure PVP.

51. The device of any one of Examples 37 to 50, wherein the blood pressure comprises central venous pressure CVP.

52. The device of any one of Examples 37 to 51, wherein the processor is configured to determine a PVP and based, on the PVP, determine a CVP.

53. The device of Example 52, further comprising a display carried by the housing, and wherein the processor is configured to output both the determined PVP and the determined CVP on the display.

54 The device of any one of Examples 37 to 53, further comprising a third sensor carried by the housing, wherein the third sensor comprises at least one of an antenna sensor, a capacitive sensor, a resistive sensor, a bioimpedance sensor, a piezoelectric sensor, an ECG sensor, and/or an optical sensor.

55. The device of any one of Examples 37 to 54, further comprising a power source.

56. The device of any one of Examples 37 to 55, further comprising a wireless transmitter.

57. The device of any one of Examples 37 to 56, wherein the processor is disposed within the housing.

58. The device of any one of Examples 37 to 57, wherein the processor is a separate device that is wirelessly coupled to the device.

59. The device of any one of Examples 37 to 58, further comprising an input device carried by the housing and coupled to the processor.

60. The device of any one of Examples 37 to 59, wherein the pressure sensor is carried by the housing.

61. The device of any one of Examples 37 to 59, wherein the position sensor is carried by the housing.

62. A device for measuring intravascular pressure, the device comprising:
a housing configured to be secured to a patient's body;
an output device carried by the housing;
a pressure sensor configured to obtain pressure data;
a position sensor configured to obtain position data;
at least one processor in communication with the pressure sensor and the position sensor;
at least one tangible, non-transitory computer-readable medium; and
program instructions stored on the at least one tangible, non-transitory computer-readable medium that, when executed by the at least one processor, cause the device to perform functions comprising:
   determine a blood pressure of the patient based on the pressure data and the position data; and
   output the blood pressure via the output device.

63. The device of Example 62, wherein the position sensor is configured to measure the position of the housing with three degrees of freedom.

64. The device of Example 62, wherein the position sensor is configured to measure the position of the housing with six degrees of freedom.

65. The device of Example 62, wherein the position sensor is configured to measure the position of the housing with nine degrees of freedom.

66. The device of any one of Examples 62 to 65, wherein the position sensor comprises an accelerometer.

67. The device of any one of Examples 62 to 66, wherein the position sensor comprises a gyroscope.

68. The device of any one of Examples 62 to 67, wherein the position sensor comprises a magnetometer.

69. The device of Example 62, wherein the position sensor comprises an inertial measurement unit.

70. The device of any one of Examples 62 to 69, wherein the output device comprises a display carried by the housing, and wherein the processor is coupled to display and configured to output the determined blood pressure on the display.

71. The device of Example 70, wherein the determined blood pressure is output as a number.

72. The device of Example 70 or Example 71, wherein the determined blood pressure is not displayed as a waveform.

73. The device of any one of Examples 70 to 72, wherein the processor is configured to output instructions for a user on the display.

74. The device of any one of Examples 62 to 73, wherein the blood pressure comprises mean venous pressure.

75. The device of any one of Examples 62 to 74, wherein the blood pressure comprises peripheral venous pressure PVP.

76. The device of any one of Examples 62 to 75, wherein the blood pressure comprises central venous pressure CVP.

77. The device of any one of Examples 62 to 76, wherein the processor is configured to determine a PVP and based, on the PVP, determine a CVP.

78. The device of Example 52, further comprising a display carried by the housing, and wherein the processor is configured to output both the determined PVP and the determined CVP on the display.

79. The device of any one of Examples 62 to 78, further comprising a third sensor carried by the housing, wherein the third sensor comprises at least one of an antenna sensor, a capacitive sensor, a resistive sensor, a bioimpedance sensor, a piezoelectric sensor, an ECG sensor, and/or an optical sensor.

80. The device of any one of Examples 62 to 79, further comprising a power source.

81. The device of any one of Examples 62 to 80, further comprising a wireless transmitter.

82. The device of any one of Examples 62 to 81, wherein the processor is disposed within the housing.

83. The device of any one of Examples 62 to 82, wherein the processor is a separate device that is wirelessly coupled to the device.

84. The device of any one of Examples 62 to 83, further comprising an input device carried by the housing and coupled to the processor.

85. The device of any one of Examples 62 to 84, wherein the pressure sensor is carried by the housing.

86. The device of any one of Examples 62 to 85, wherein the position sensor is carried by the housing.

87. A method for determining blood pressure, the method comprising:
receiving first position data from a position sensor carried by a device secured to a patient's body;
receiving pressure data from a pressure sensor carried by the device, wherein the pressure data characterizes a blood pressure of the patient;
based at least one the pressure data, determining a blood pressure of the patient;
receiving second position data from the position sensor; and
comparing the second position data to the first position data and,
   if a difference between the first and second position data exceeds a predetermined threshold, indicating to a user via the device that the patient and/or device is out of position and/or should be repositioned, and
   if a difference between the first and second position data does not exceed a predetermined threshold, indicating to a user via the device that a blood pressure measurement may be taken.

88. The method of Example 87, wherein the data is first pressure data taken at a first time and the method comprises taking second pressure data at a second time after the first time while the device is in a position characterized by the second position data.

89. The method of Example 88, wherein the blood pressure measurement is a first blood pressure measurement and the method further comprises determining a second blood pressure measurement based on the second pressure data and the difference between the first and second position data.

90. The method of Example 89, further comprising displaying the second blood pressure measurement via an output device carried by the device.

91. A method for determining blood pressure, the method comprising:
receiving first position data from a position sensor carried by a device secured to a patient's body;
receiving first pressure data from a pressure sensor carried by the device, wherein the first pressure data characterizes a blood pressure of the patient;
based at least one the first pressure data, determining a first blood pressure of the patient;
receiving second position data from the position sensor;
comparing the second position data to the first position data;
receiving second pressure data from the pressure sensor; and
determining a second blood pressure of the patient, wherein the second blood pressure is based on the second pressure data and the difference between the first position data and the second position data.

92. A peripheral venous pressure monitoring patch device comprising:
a single watertight housing encompassing a fluid-filled pressure-transducer, electronic components, and external display;
an adhesive housing docking bracket, configured to be placed approximately in the 4th intercostal space, mid-anterior/posterior chest wall, for minimum of 24 hours and up to 14 days;
an IMU disposed within the housing facilitating the detection of the position of the patient and detect respiratory cycle, heart rate, and/or stroke volume;
an automatic zeroing function in which the pressure transducer is automatically zeroed via a function that is coupled to turning on the device once the device has been tethered to patient via adhesive patch;
a software implementation that guides users through each step of the preparation and measurement process; and
a signal processing unit that permits measurement and display of peripheral venous pressure at end-expiration, only after zeroing and other pre-measurement steps have been satisfied.

93. A non-invasive device for measuring a patient's blood pressure, the device comprising:
a housing configured to be worn on the body of the patient,
a first port in the housing, the first port configured to be detachably coupled to an outlet end of a fluid source;
a second port in the housing, the second port configured to be detachably coupled to a proximal end of a catheter;
a fluid passageway extending from the first port through the housing to the second port such that the first and second ports are fluidly coupled via the fluid passageway and such that, when the first port is coupled to the fluid source and the distal end of the catheter is disposed in a patient's blood vessel, a fluid from the fluid source flows through the fluid passageway and into the blood vessel;
a pressure sensor carried by the housing, wherein the pressure sensor is operatively coupled to the fluid passageway and configured to measure a fluid pressure along the passageway;
a position sensor carried by the housing, wherein the position sensor comprises a magnetometer and at least one of a gyroscope and an accelerometer, and wherein the position sensor is configured to determine the absolute position of the housing relative to an initial reference position; and
a controller coupled to the pressure sensor and the position sensor, wherein the controller comprises memory and processing circuitry configured to determine a blood pressure of the patent via data received from the pressure sensor and/or the position sensor.

94. The device of Example 93, wherein the controller is configured to determine a peripheral venous pressure.

95. The device of Example 93 or Example 94, wherein the controller is configured to determine a central venous pressure.

96. The device of any one of Examples 93 to 95, wherein the device includes a display configured to display to a user a pressure measurement.

97. The device of Example 96, wherein the controller is configured to cause the display to display instructions to the user.

98. The device of any one of Examples 93 to 97, wherein the device is powered by a battery contained within the housing.

99. The device of any one of Examples 93 to 98, wherein the device includes an inertial measurement unit (IMU) sensor with 6 degrees of freedom.

100. The device of any one of Examples 93 to 98, wherein the device includes an inertial measurement unit (IMU) sensor with 9 degrees of freedom.

101. The device of any one of Examples 93 to 100, wherein the device is configured to measure a patient's blood pressure while positioned on the patient's skin.

102. The device of any one of Examples 93 to 101, wherein the controller is disposed within the housing.

103. The device of any one of Examples 93 to 102, wherein the controller is a separate device that is wirelessly coupled to the device.

104. A non-invasive device for measuring a patient's blood pressure, the device comprising:
a housing configured to be worn on the body of the patient,
a first port in the housing, the first port configured to be detachably coupled to an outlet end of a fluid source;
a second port in the housing, the second port configured to be detachably coupled to a proximal end of a catheter;
a fluid passageway extending from the first port through the housing to the second port such that the first and second ports are fluidly coupled via the fluid passageway and such that, when the first port is coupled to the fluid source and the distal end of the catheter is disposed in a patient's blood vessel, a fluid from the fluid source flows through the fluid passageway and into the blood vessel;
a pressure sensor carried by the housing, wherein the pressure sensor is operatively coupled to the fluid passageway and configured to measure a fluid pressure along the passageway;
a position sensor carried by the housing, wherein the position sensor is configured to measure the position of the housing with 9 degrees of freedom; and
a controller coupled to the pressure sensor and the position sensor, wherein the controller comprises memory and processing circuitry configured to determine a blood pressure of the patent via data received from the pressure sensor and/or the position sensor.

105. A system comprising:
a housing configured to be affixed to a patient's body, the housing including an indicator;
a pressure sensor configured to obtain pressure data;
a position sensor configured to obtain position data;
at least one processor in communication with the pressure sensor and the position sensor;
at least one tangible, non-transitory computer-readable medium; and
program instructions stored on the at least one tangible, non-transitory computer-readable medium that, when executed by the at least one processor, cause the system to perform functions comprising:
   based on position data received from the position sensor, indicate to a user that the housing and/or patient is in position for a pressure measurement or that the housing and/or patient needs to be adjusted, and
   based on pressure data received from the pressure sensor, determine a blood pressure of the patient and cause the indicator to indicate to the user the blood pressure of the patient.

106. The system of Example 105, wherein the indicator is one of a display with text and/or numbers, an LED, and/or a speaker.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 schematically depicts a device for measuring blood pressure configured in accordance with several embodiments of the present technology.

FIGS. 2A and 2B show a device for measuring blood pressure secured to a patient's body and ready to take a pressure measurement, in accordance with several embodiments of the present technology.

FIG. 3 is a flow chart showing a method for using a device of the present technology to measure a patient's blood pressure.

FIG. 4A is a top view of a device for measuring blood pressure in accordance with several embodiments of the present technology.

FIG. 4B is a perspective view of the device shown in FIG. 4A.

FIG. 5A shows a docking structure for use with the blood pressure measuring devices of the present technology.

FIG. 5B shows the docking structure in the process of being coupled to a device for measuring blood pressure configured in accordance with several embodiments of the present technology.

FIG. 5C is an enlarged cross-sectional view of a coupling region between the device and the docking structure.

FIGS. 6A and 6B are cross-sectional side and cross-sectional axial views, respectively, of a device for measuring blood pressure configured in accordance with several embodiments of the present technology.

FIG. 7 is a block diagram of a device for measuring blood pressure configured in accordance with several embodiments of the present technology.

FIG. 8 shows an example method for using the monitoring device in accordance with the present technology.

FIG. 9 is a table showing example user interface prompts of a monitoring device configured in accordance with the present technology.

FIGS. 10, 11, and 12 show clinical applications of the blood pressure measuring devices of the present technology.

### DETAILED DESCRIPTION

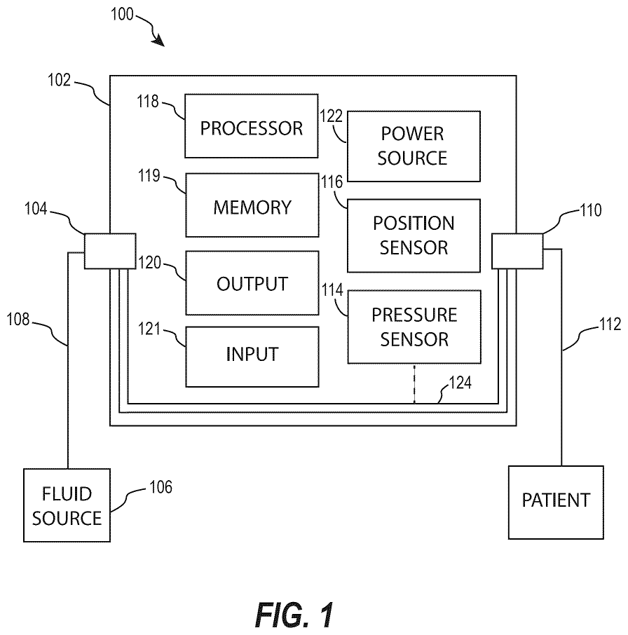

The present technology relates to devices and methods for non-invasive measurement of one or more physiological parameters of a patient, and in some examples, to devices and methods for non-invasive measurement of venous pressure. The monitoring devices disclosed herein can be used by lower-skilled care providers (e.g., a registered nurse, a registered practical nurse, etc.) in lower-resource settings (for example, hospital wards, general medicine floors, clinics, etc.) is described. Specific details of several embodiments of the technology are described below with reference to FIGS. 1-12.

FIG. 1 schematically depicts a blood pressure measurement device 100 (or “device 100”) configured in accordance with several embodiments of the present technology. The device 100 can comprise a housing 102 having a first port 104 configured to be fluidly coupled to a fluid source 106 (e.g., directly or via tubing 108), a second port 110 configured to be detachably coupled to an end of a cannula 112, and a fluid channel 124 extending from the first port 104 through the housing 102 to the second port 110 such that the first and second ports 104, 110 are fluidly coupled via the fluid channel 124. The first port 104 may be fluidly coupled to the fluid source 106 while the other end of the cannula 112 is disposed within a patient's blood vessel (e.g., a vein), thereby enabling fluid flow from the fluid source 106, through the fluid channel 124 and cannula 112 and into the patient's blood vessel. The device 100 may further include a pressure sensor 114, a position sensor 116, a processor 118, the memory 119, one or more output devices 120, one or more input devices 121, and a power source 122, all carried by the housing 102. In some embodiments, all of the pressure sensor 114, the position sensor 116, the processor 118, the memory 119, the one or more output devices 120, the one or more input devices 121, and the power source 122 are fully enclosed by the housing 102. In other embodiments, at least some of the foregoing embodiments are not fully enclosed by the housing 102.

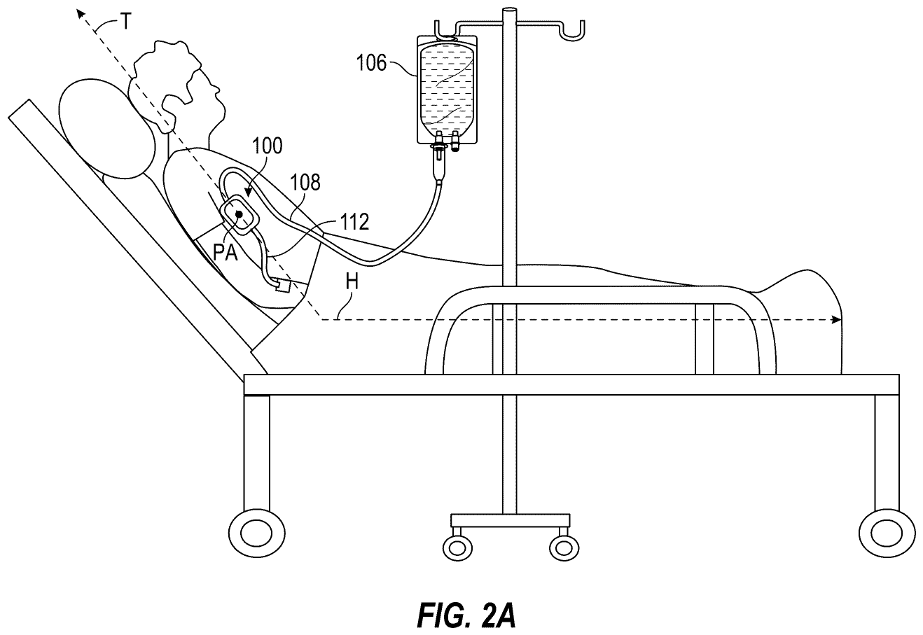

As shown in FIG. 2A, the device 100 is configured to be secured to a patient's body to obtain a blood pressure measurement. In some examples, the device 100 is sized and shaped for placement at the phlebostatic axis PA of the patient, as the phlebostatic axis PA is vertically aligned with the right atrium of the heart and most accurately reflects a patient's hemodynamic status. The care provider can locate and/or approximate the phlebostatic axis at the lateral aspect of the chest or abdomen, half of an inch between the anterior-posterior mid-line, or in the third or fourth intercostal space at the mid-anterior-posterior diameter of the chest wall. The device 100 may include an adhesive patch at a bottom surface of the housing 102 to enable securement to the chest wall. In some embodiments, the device 100 is secured to the patient via a docking structure that is adhered to the patient via an adhesive patch, as discussed in greater detail herein. In any case, the device 100 is configured to use the device position data obtained by the on-board position sensor 114 to instruct (or otherwise give feedback to) the care provider to position the device 100 at the same location on the patient's body and/or position the patient's torso at the same height for each blood pressure measurement, thereby eliminating or reducing the effects of hydrostatic pressure between measurement and improving consistency across multiple readings. As described in greater detail below, in some embodiments the position data may be used to calibrate the blood pressure measurement so that the device 100 and/or patient need not be in the same position for each measurement.

In FIG. 2A, the device 100 is shown with the first port 104 fluidly coupled a fluid source 106 (e.g., an IV bag) while the second port 110 is fluidly coupled to a cannula 112 inserted into the patient's antecubital vein located in the crease of the elbow. It will be appreciated that other cannula insertion locations are possible. FIG. 2B depicts another use case in which the first port 104 remains unconnected to a fluid source 106 while the second port 110 is fluidly coupled to the cannula 112.

Referring to FIGS. 1-2B together, in some embodiments the first port 104 (labeled in FIG. 1 only) includes a coupling structure configured to mate with a fluid source 106 and/or one or more intermediate connecting elements, such as tubing 108 and/or one or more intermediate connectors. The fluid source 106 may be a syringe, an intravenous bag (an “IV bag”), or other suitable fluid sources. Additionally or alternatively, the first port 104 may be configured to be coupled to a neutral displacement connector (e.g., a needleless valve connector and others). The second port 110 can include a coupling structure configured to mate with a cannula 112 and/or one or more intermediate connecting elements, such as tubing and/or one or more intermediate connectors. One or both of the first and second ports 104, 110 can include or be, for instance, a Luer adapter (e.g., a spin Luer, a split Luer, etc.), taper, collar, slip, connector, lock structure, or other coupling structure. Each of the first and second ports 104, 110 can carry a removable or pierceable/penetrable end cap or seal (not shown) to facilitate the maintenance of a controlled environment within the device 100.

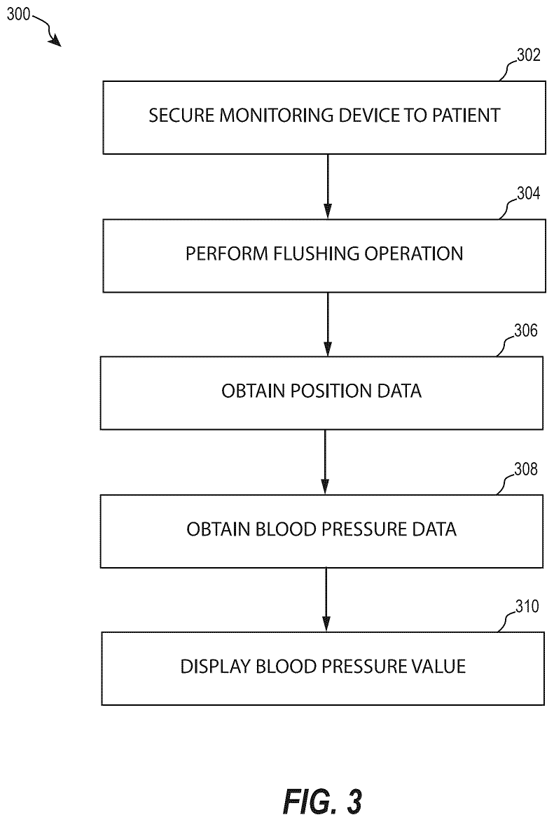

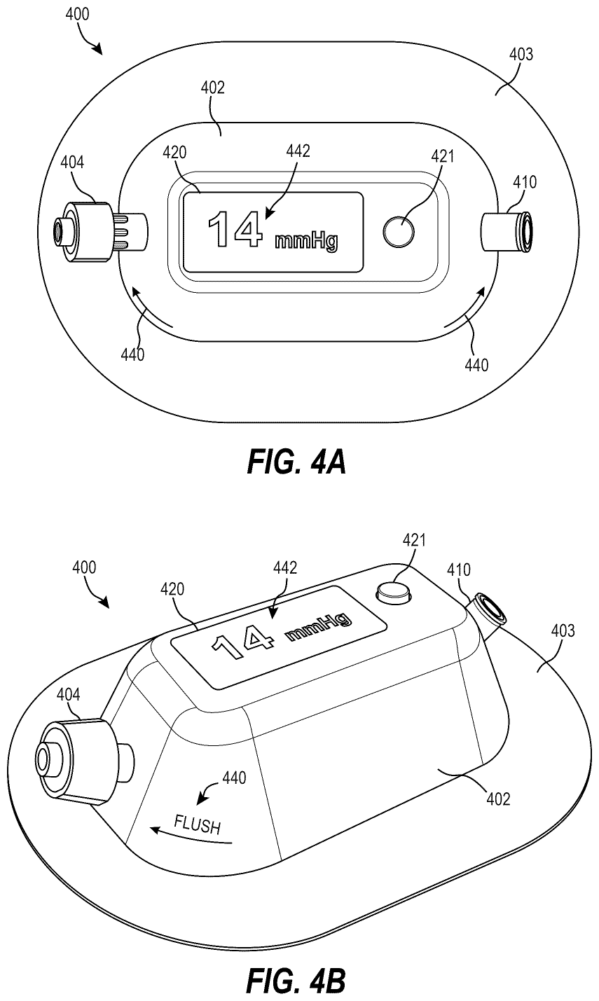

The housing 102 may optionally include one or more markings to identify various features of the device 100, such as an arrow and/or text to indicate which of the ports should be used for a flushing operation and/or an arrow and/or text to indicate which of the ports should be coupled to the catheter and/or another external device (see, for example, FIGS. 4A and 4B).

The device 100 may further include a pressure sensor 114 carried by the housing 102 and configured to output signals (e.g., pressure data and/or pressure waveforms) to the processor 118 and/or the memory 119 on a continuous or periodic basis. The pressure sensor 114 may be operatively coupled to the fluid channel 124 and configured to measure a fluid pressure within and/or along the channel and/or a portion of the channel, which is used to approximate the blood pressure within the blood vessel in which the cannula 112 is placed. The channel 124 can include one or more openings, windows, or ports to facilitate direct access to or physical contact with a substance carried within the channel 124. In some embodiments, the pressure sensor 114 can include a fluid-filled pressure sensor, such as a piezoelectric pressure transducer coupled to a diaphragm and/or flexible membrane with one side exposed to an opening in the channel 124 (and thus the pressure source being measure) and the other side exposed to atmospheric pressure. When the channel 124 is in fluid communication with blood sourced from a vessel, vascular pressure exerts a displacement force upon the diaphragm, causing the diaphragm to deflect and/or stretch, causing a corresponding resistance change. This change in resistance generates an electrical signal corresponding to an instantaneous, quasi-instantaneous, or near-instantaneous vessel pressure reading. According to some implementations, the fluid-filled sensor includes a capacitive sensor used in a similar fashion to the piezoelectric transducer. One capacitive element may be positioned on the diaphragm and separated from another capacitive element by some distance. As the diaphragm deflects the distance between the capacitive elements changes, therefore changing the capacitance and being measured as a corresponding pressure.

In those embodiments including a fluid-filled pressure sensor, the device 100 may optionally be configured to be coupled to a fluid source to prime the fluid-filled pressure transducer. In some embodiments, the fluid source may include a saline flush (syringe) of 5 mL, 10 mL or 20 mL volume. In some embodiments, the fluid source may consist of a bag of saline. In some embodiments, the device 100 may include a reservoir (not shown) carried by the housing 102 and operatively coupled to the fluid channel 124. The reservoir may be filled by the care provider with saline with volumes such as 5 mL, 10 mL, 15 mL, or 20 mL. In these and other embodiments, the device 100 may include a flush tab operably coupled to the reservoir that allows for release of fluid into the pressure tubing system into the patient when pulled. In some embodiments, the sensors and algorithms within the device 100 can be used to sense whether the device was successfully flushed and convey that information back to the user, as discussed in greater detail below. Other sensing devices may be used to measure displacement of the diaphragm, such as a surface acoustic wave (SAW) sensor, a solid state sensor, a mechanical sensor, a potentiometric sensor, and a resonant wire sensor.

In some implementations, the device 100 includes a valve carried by the housing and operably coupled to the pressure sensor 114 and/or channel 124. The valve can be configured to intermittently seal and/or open the end of the pressure sensor 114 that is exposed to air during a zeroing operation (as described herein). The valve can move between the open and closed positions either by manual (e.g., via an input device 121 at the exterior of the housing 102), electric, pneumatic, or hydraulic actuation. In some embodiments, a zeroing operation is performed by pushing a button on the external surface of the housing 102 that forces the valve to open, thereby exposing the pressure sensor 114 to air while simultaneously closing off exposure of the pressure sensor 114 to the channel 124 (and then blood therein). To close the valve, the button may be released.

The position sensor 116 is configured to obtain position data of the device 100 to ensure blood pressure measurement consistency across multiple readings. The position sensor 116 may be configured to detect the absolute position and orientation of the device 100 relative to an initial reference position, thereby enabling compensation for hydrostatic pressure variations that might otherwise affect measurement accuracy. In some examples, the position sensor may comprise a 9 degree-of-freedom (dof) inertial measurement unit (IMU) incorporating an accelerometer, a gyroscope, and a magnetometer working in concert to determine absolute positioning in three-dimensional space. The IMU may include its own processor and/or memory, one or both of which may be operably coupled to the device processor 118 and configured to send position data to the device processor 118.

In some implementations, the position sensor 116 is configured to obtain position data with 6 dof. In such embodiments, the position sensor 116 may comprise an accelerometer and a gyroscope, an accelerometer and a magnetometer, a gyroscope and a magnetometer, and other suitable sensors arrangements. In yet other examples, the position sensor 116 is configured to obtain position data with 3 dof. In such embodiments, the position sensor 116 may comprise an accelerometer, a gyroscope, or a magnetometer. The position sensor 116 can be coupled to the processor 118 and is configured to output signals (e.g., position data) to the processor 118 and/or the memory 119 on a continuous or periodic basis.

By securing the device 100 at the same location along the chest wall (e.g., at the phlebostatic axis) for each measurement session, the device's position relative to the chest wall remains constant. When a patient's position changes between measurement sessions, however, the resulting hydrostatic pressure differences may obscure interpretation of the blood pressure readings. For example, with reference to FIG. 2A, if an initial blood pressure measurement is taken with the patient's torso T at 15 degrees relative to horizontal H and a subsequent measurement is taken with the patient's torso T at 50 degrees relative to horizontal H, the device 100 will be at a higher elevation in the second reading. Thus, even if the patient's clinical and/or physiological status is the same during the first and second measurements, the second measurement will be lower due to hydrostatic pressure. The position sensor 116 may address this concern by providing positional context for each measurement, allowing care providers to distinguish between changes in readings caused by patient positioning versus those resulting from actual physiological changes (such as changes in cardiac hemodynamics or fluid status).

In some cases, the device 100 may be configured to compare current device position data with previously recorded reference positions. In some implementations, when the device 100 detects a positional discrepancy exceeding predetermined thresholds, the device 100 alerts the care provider through the one or more output devices 120. As detailed herein, such alerts may include visual guidance displayed on a display carried by the housing 102 and provide guidance to the care provider on how to reposition the patient to match the reference position established during the initial measurement. In certain examples, the position data may be used to mathematically compensate for known hydrostatic effects without requiring physical repositioning of the patient, thereby enhancing measurement convenience while maintaining accuracy. For example, the patient's measured position may be used in conjunction with the initial or previous reference position and hydrostatic pressure to compute the expected offset in pressure.

The integration of a magnetometer with the accelerometer and gyroscope may provide substantial benefits in maintaining accurate positional awareness over extended periods. Such a sensor array may overcome limitations associated with simpler position sensing systems, such as cumulative yaw drift that might otherwise introduce increasing positional errors over time. By maintaining stable positional references even in dynamic clinical environments, the position sensor 116 may contribute significantly to measurement reliability. Furthermore, the position data may be stored in the device memory 119 along with corresponding pressure measurements, enabling retrospective analysis of positional factors that might influence measurement interpretation and potentially revealing clinically relevant trends that might otherwise remain obscured by positional artifacts.

Instead of or in addition to the position sensors detailed above, the device 100 may rely on one or more alternative position sensors. For example, in some implementations the device 100 includes optical fiducials carried by the housing 102 that are tracked by an optical vision system in the patient's room. In some implementations, the device 100 may include electromagnetic (EM) trackers carried by the housing 102 and configured to communicate with an electromagnetic tracking system in the patient's room. In these and other embodiments, the device 100 may include a barometric pressure sensor carried by the housing 102 and configured to measure a vertical height of the device 100. Additionally or alternatively, the device 100 may include one or more temperature sensors carried by the housing 102 and/or one or more humidity sensors carried by the housing 102 and configured to measure environmental variables that may be used to make corrections to other sensor measurements. For example, in some implementations, the temperature data can be used to correct errors in the position and/or pressure data and/or parameters derived from the data.

In some embodiments, the position sensor 116 enables assessment of the patient's respiratory cycle, as the position data obtained by the position sensor 116 can be evaluated to detect movement of the device 100 corresponding to chest wall movements associated with breathing. The processor 118 may utilized the position and/or movement data (received from the position sensor 116) to determine the timing of expiration and inspiration and select blood pressure measurement that occurs at the end of expiration for display to the care provider. This may be beneficial as PVP can vary with inspiration and expiration (e.g., PVP decreases with inspiration), and thus it may be desirable to ensure measurements are consistently taken at the same time within the respiratory cycle for adequate control, when comparing sequential measurements. Such methods can be especially advantageous when using PVP as a method for adjusting diuretic doses in heart failure, and either increasing, decreasing or stopping diuretics based on a change of PVP or achieving a target PVP.

In some implementations, the position sensor 116 enables the collection of ballistocardiogram and/or cardioseismography measurements that provide information about heart rate, heart rate variability, and stroke volume, and that can be used in conjunction with PVP to provide important cardiac monitoring metrics that may be relevant for managing a patient admitted to hospital with acute or chronic heart failure. In some examples, PVP measurements may be coupled with ballistocardiogram and/or cardioscismography measurements to obtain estimates of cardiac filling pressures such as pulmonary artery (systolic or diastolic) or left atrial or left ventricular diastolic pressures. In some examples, an artificial intelligence algorithm may be developed using PVP and one of cardioseismography or ballistocardiogram as obtained from the position sensor. In some examples, the artificial intelligence algorithm may be further developed by inputting and comparing the device inputs with invasive cardiac filling pressures as measured by cardiac catheterization.

In some embodiments, the position sensor 116 can be used to sense movements that relate to various stages of general use and can be used in the logic structure to prompt users. For example, the position sensor 116 can be configured to sense coupling with the docking structure by sensing the movement(s) associated with engagement with the docking structure (in addition to or instead of detecting coupling with the proximity sensor). Additionally or alternatively, the position sensor 116 can be configured to sense coupling of an external device (e.g. syringe, tubing, another connector, etc.) to the first and/or second port 104, 110 by sensing the movement(s) associated with engagement of the first and second ports 104, 110. Other examples are possible. In such embodiments, the position sensor 116 can communicate the position data to the processor 118, and the processor 118 is configured to analyze the position data (according to one or more algorithms) to determine whether a predetermined device activity (e.g., docking, connection of one or both ports, etc.) has occurred. If the processor 118 determines a certain activity has occurred, the processor 118 can communicate to the user (e.g., via the one or more output devices) that a certain activity has occurred and/or prompt the user to take a next step in the method.

The device 100 may optionally include one or more other sensors in addition to the pressure and position sensors 114, 116. For example, the device 100 can one or more light emitting diodes (LEDs), impedance sensor, an ECG sensor (for measuring electrical activity), an electrode, capacitive sensor, resistive sensor, semiconductor lasers, optical detectors (e.g., photodiodes, which can be configured to detect optical signal characteristics such as intensity, peak wavelength, or phase shift), temperature sensors (e.g., an optical temperature sensor or a thermocouple), fluid flow sensors (e.g., a Doppler ultrasound transducer and detector), substance or environment sensing field effect transistors (e.g., a chemical sensing or chemically modified FET (ChemFET), an ion sensitive FET (ISFET), an Enzyme modified FET (EnFET), or an electrolyte-oxide-semiconductor FET (EOSFET)), an electrophoresis device, and/or other sensing elements or devices.

In some implementations, the device 100 may be configured to obtain data indicative of the electrical activity of the patient's heart. For example, the device 100 can include an electrode carried on the bottom of the housing 102 and configured to be placed in contact with the patient's skin. The device 100 may further include one or more ports for detachably coupling one or more additional electrode leads to the housing 102, thereby placing the additional electrode lead(s) in electrical communication with the processor 118. The additional electrode(s) can be positioned elsewhere on the patient (such as on the chest, arms, or legs). In some embodiments, the device 100 does not include an electrode integrated with the housing 102 and instead is configured only to be electrically coupled to external electrodes. The processor 118 may be configured to process the electrical signals of the heart, which may be useful combination with PVP measurements. For example, measuring ECG/electrical activity may allow for the detection of specific aspects of a venous waveform corresponding to specific phases of the cardiac cycle such as atrial contraction, closure of tricuspid valve, and specific pathologies such as but not limited to exaggerated v-waves (tricuspid regurgitation) and prominent X and Y descents (pericardial constriction).

According to some embodiments, the device 100 is configured to obtain photoplethysmography (PPG) data. For example, the device 100 can include a light source (such as an LED) and a photodetector configured to measure the amount of light reflected or transmitted through the tissue. The light source and photodetector may be carried by the housing 102 and positioned at a bottom surface of the device 100 such that the light source and photodetector are adjacent the patient's skin. The processor 118 may be configured to process the blood pressure measurements along with the PPG data and/or ECG data to estimate cardiac filling pressures. In some embodiments, PPG data, ECG data, and/or seismocardiography data may be processed along with the PVP data and may be compared (e.g., via AI) to gold standard central venous pressure and/or pulmonary artery pressure (e.g., measured directly from a sensor positioned in the patient's jugular or subclavian vein, or directly from the patient's pulmonary artery) to determine a relationship to better estimate CVP and/or pulmonary artery pressure from PVP and the aforementioned one or more physiological measurements.

In some examples, the device 100 is configured to obtain blood oxygenation data. For example, the device 100 can include a light source (e.g., a visible LED and at least one infrared LED) and a photodetector, both carried by the housing 102 and positioned adjacent the channel 124. The light source may be configured to emit optical signals at or centered about particular wavelengths (e.g., approximately 660 nm, and one or more of approximately 905, 910, and 940 nm) into the channel 124 and the photodetector is configured to detect the transmitted optical signals. Optical signal absorption by blood or another substance in the channel 124 affects the transmitted intensity of such signals. Based upon known oxyhemoglobin and/or deoxyhemoglobin absorbance spectra corresponding to particular optical wavelengths, the processor 118 can determine a blood oxygenation level or state.

The processor 118 may be configured to process input data according to instructions stored in memory 119. The processor 118 can include a state machine, a microcontroller, a microprocessor, an application specific integrated circuit (ASIC), or a field programmable gate array (FPGA) or programmable logic device (PLD) configured to correspond to or execute program instruction sequences (e.g., software and/or firmware) directed to receiving, operating upon, evaluating, analyzing, interpreting, and/or transforming signals generated by one or more of the sensors (e.g., the pressure sensor 114, the position sensor 116, etc.), and determining one or more physiological parameters (e.g., blood pressure, venous blood pressure, arterial blood pressure, PVP, central venous pressure, heart rate, respiratory rate, etc.). The processor 118 may then display a single pressure measurement via the display, which advantageously allows a lower skilled care provider (for example, operating on a general medical floor) to obtain a measure of blood pressure without having to interpret blood pressure waveforms.

In some embodiments, the processor 118 is configured to generate a mean value of blood pressure (e.g., venous blood pressure, arterial blood pressure, PVP, central venous pressure, etc.). using a time series of pressure data generated by the pressure sensor 114. Additionally or alternatively, the processor 118 can generate a maximum or mean value of blood pressure fluctuation, range, amplitude, or magnitude using this time series of pressure data. As a representative example, the processor 118 can average a series of sensed instantaneous vascular pressure values to determine a mean vascular pressure value with respect to a predetermined time period (e.g., approximately 1-10 seconds, 30 seconds, 1 minute, or longer). The processor 118 can additionally or alternatively determine a maximum and/or average vascular pressure fluctuation value relative to a predetermined time period. As discussed herein, the processor 118 may display the blood pressure value as a number and not as a waveform. Providing a blood pressure value, rather than a waveform, can be beneficial as viewing a number (versus interpreting a waveform) is simpler and faster for the care provider.

In some examples, a coefficient or regression formula (such as a linear or non-linear regression) may be applied to a measured PVP in order to more accurately estimate central venous pressure of a patient with heart failure. A coefficient or regression formula may be developed after completing clinical studies in which invasive PVP data is compared with central venous pressure data. The derivation of the coefficient or regression formula may include but is not limited to factors such as heart rate, age, sex, weight, temperature, EKG rhythm, respiratory rate as examples.

In some embodiments, the processor 118 comprises a processor carried by the housing 102 and a second processor, separate from the device 100. In such cases, data obtained by the sensor(s) and/or processor 118 may be communicated to the remote processor through a wired or wireless connection.

The memory 119 may be a tangible, non-transitory, computer-readable medium configured to store instructions that are executable by the processor 118. For example, the memory 119 may be data storage loaded with software code that is executable by the processor 118 to achieve certain functions. The memory 119 can include an electronically or computer programmable or readable medium having one or more of a Random Access Memory (RAM), a Read Only Memory (ROM) such as a type of programmable ROM (PROM), a set of registers, or other data storage elements for storing a) program instruction sequences; b) signals generated or output by the one or more sensors or physiologic parameter values corresponding thereto; and c) reference data that facilitates the determination, evaluation, or analysis of sensed physiologic parameter values. In some embodiments, the memory 119 comprises a memory carried by the housing 102 and one or more remote memories, separate from the device 100. In such cases, data obtained by the sensor(s) and/or processor 118 may be communicated to the remote memory through a wired or wireless connection.

The foregoing discussion provides a brief, general description of a suitable environment in which the present technology may be implemented. Although not required, aspects of the technology are described in the general context of computer-executable instructions, such as routines executed by a general-purpose computer. Aspects of the technology can be embodied in a special purpose computer or data processor that is specifically programmed, configured, or constructed to perform one or more of the computer-executable instructions explained in detail herein. Aspects of the technology can also be practiced in distributed computing environments where tasks or modules are performed by remote processing devices, which are linked through a communication network (e.g., a wireless communication network, a wired communication network, a cellular communication network, the Internet, a short-range radio network (e.g., via Bluetooth)). In a distributed computing environment, program modules may be located in both local and remote memory storage devices.

The power source 122 may include a battery to power the electrical components, such as the processor(s) 118, the memory 119, the pressure sensor 114, the position sensor 116, etc. In some implementations the power source 122 includes a power interface configured to receive current and thereby charge the battery. The power interface may include a direct current (DC) interface such as a USB connection (e.g., USB Type-C), as well as other suitable connections.

The device 100 may optionally include a wireless transmitter (e.g., Wi-Fi, Bluetooth, etc.), that sends raw data obtained by one, some, or all of the sensors on the device to a remote signal processing unit on a mobile or stationary device such as a phone, tablet, or computer. In some embodiments, the wireless transmitter subsequently receives the processed data for display on the device 100 itself, or in some embodiments, the processed data is displayed on the remote device (in addition to or instead of display on the device).

As previously mentioned, the device 100 may include one or more output devices 120 configured to communicate information to care providers, such as physiological data (e.g., blood pressure, heart rate, etc.), information regarding device maintenance or functional status, and/or instructions to aid the care provider in using the device 100. The output device(s) 120 may be positioned at a top surface of the housing 102 (as shown in FIG. 4A, for example) and/or or at any of the sidewalls of the housing 102.

In some examples the device 100 includes a digital display positioned at an exterior surface of the housing 102 and configured to display numerical blood pressure values and instructions for the care provider. In some examples, the display is configured to display a variety of clinical information including but not limited to venous pressure, PVP, central venous pressure, pulmonary pressure, left atrial pressure, ECG heart rhythm, oxygen saturation, heart rate, temperature, and/or arterial blood pressure.

In some examples, the display may comprise a liquid crystal display (LCD), while in other implementations, the display may comprise an organic light-emitting diode (OLED) display or other suitable display technology providing appropriate visibility in various clinical lighting conditions.

In some implementations, the display may present information through various visual modalities to enhance comprehension and accommodate different user preferences. Textual information may be displayed in adjustable font sizes and styles to ensure readability in diverse clinical environments, while numerical values may be presented with appropriate units of measurement and reference ranges to facilitate rapid interpretation. Additionally or alternatively, the display may utilize graphical representations including trend lines, bar graphs, or waveforms to illustrate blood pressure patterns over time, enabling care providers to identify significant changes or cyclic variations. The display may incorporate a range of symbols and icons designed to convey status information efficiently, such as battery level indicators, connection status symbols, and alert notification icons. In certain implementations, color-coding may be employed throughout the interface wherein different colors may represent various pressure ranges or alert thresholds—for example, measurements within normal parameters may appear in green text, while elevated readings may appear in yellow or red depending on severity. The display may further implement contrast adjustments and night-mode options to maintain visibility across varying ambient lighting conditions common in healthcare settings, ensuring that vital information remains accessible to care providers at all times without causing eye strain or disturbing patients during nighttime monitoring sessions.

In addition to or instead of the display, the device 100 may incorporate one or more indicator lights positioned at visible locations on the housing 102. Such indicator lights may include light-emitting diodes (LEDs) of various colors, where each color may be associated with a particular status or alert condition. For example, a green light may indicate normal operation, an amber light may indicate a non-urgent alert condition, and a red light may indicate an urgent alert condition requiring immediate attention. Additionally or alternatively, different colors may be used to indicate whether the patient is positioned at the correct orientation. In some embodiments, the light(s) may be used in a binary manner, for example the light may only turn on or turn off when the patient is in the correct position and/or upon achieving a secure attachment to the dock. In some embodiments, the indicator lights may be configured to flash at various rates to convey additional information regarding the urgency of the alert condition.

The device 100 may further comprise audible output devices including one or more speakers or buzzers contained within the housing. Such audible outputs may be configured to generate sounds of varying tones, volumes, and patterns to communicate different types of information to care providers. In certain implementations, the audible outputs may be customizable to accommodate various clinical environments. The device 100 may also include tactile output mechanisms, such as vibration motors, configured to provide haptic feedback to users when holding or wearing the device, which may be particularly useful in noisy clinical environments where audible alerts might not be readily perceived.

According to some implementations, the output device 120 may comprise one or more output devices carried by the housing 102, and one or more separate and/or remote output devices that are configured to be wired to and/or wirelessly coupled to the device 100 and configured to visually, audibly, and/or haptically communicate information to the care provider. In yet additional examples, the device 100 does not include an integrated output device and instead is configured to be wired to and/or wirelessly coupled to the device 100 to display information, including a blood pressure value, to the care provider. In either case, the device 100 may wirelessly transmit data (e.g., raw measured signals, processed data, device maintenance data, and/or others) directly to a remote handheld computing device (e.g., a phone, tablet, device-specific controller, etc.) and/or desktop computing device utilized by the care provider. In some implementations, the device 100 may be configured to be coupled to the remote handheld computing device and/or desktop computing device via a wired connection.

As previously mentioned, the device 100 may further include one or more input devices 121 to enable user interaction and configuration. The input device(s) 121 may be positioned at a top surface of the housing 102 (as shown in FIG. 4A, for example) and/or or at any of the sidewalls of the housing 102. In some implementations, the device 100 may include a touchscreen display that serves as both an output and input mechanism, allowing care providers to navigate menus, enter patient information, adjust settings, and acknowledge alerts through direct touch interaction. Additionally or alternatively, the device may incorporate physical buttons, switches, analog pads, capacitive touch sensors, or dials positioned on accessible portions of the housing. These physical inputs may include power buttons, menu navigation controls, and dedicated function buttons for frequently used operations. In some embodiments, user prompts/guidance may be provided by a display on the housing 102 with requests for a user to confirm that steps have been completed via actuating a user input, such as pressing a button on the housing 102. In some embodiments, the input device 121 can include a microphone operably coupled to the processor 118 for processing the user's speech.

The device 100 may further optionally include data ports, such as USB connections, allowing direct connection to external computers or other medical equipment for data transfer, firmware updates, or advanced configuration options. In some implementations, the device 100 may also incorporate voice recognition technology, enabling hands-free operation through spoken commands, which may be particularly advantageous in scenarios where the care provider's hands are occupied with other clinical tasks.

### I. Example Methods of Use

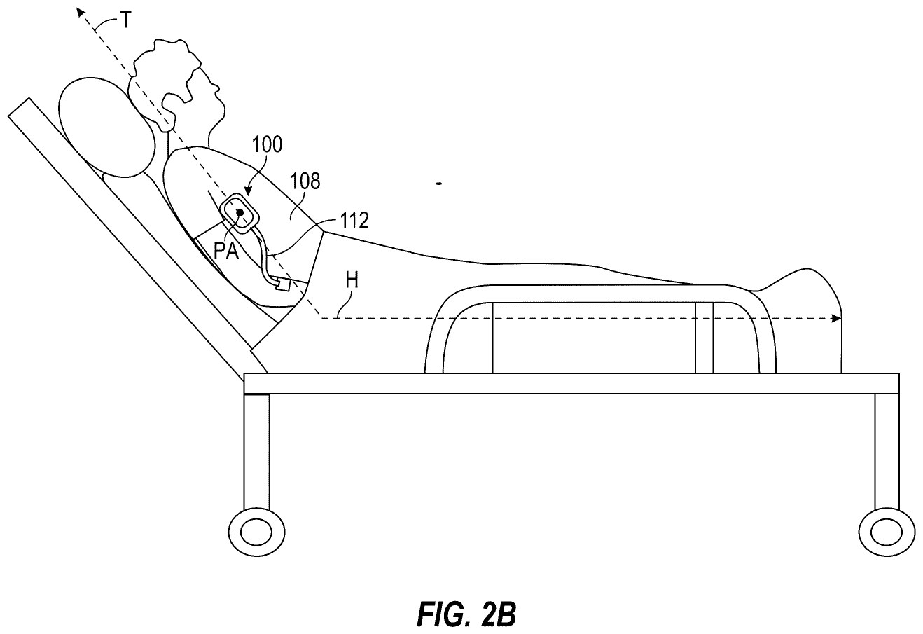

FIG. 3 is a flow diagram illustrating a method 300 for using the devices disclosed herein (e.g., device 100, 400, 600, 700, etc.) to measure a patient's blood pressure. In general, the method can comprise securing the device 100 to the patient (block 302), flushing the device 100 (block 304), obtaining position data (block 306), obtaining pressure data (block 308), and determining blood pressure value (block 310). The foregoing steps may be performed in the order provided or a different order. For example, in some methods flushing the device may occur prior to securing the device 100 to the patient. Various aspects of the method will now be described in greater detail below.

As previously mentioned, the device 100 may be placed on the patient's body at the level of the atria of the heart, which may be approximated by positioning the device 100 at the patient's phlebostatic axis. The phlebostatic axis can be identified using external landmarks, such as the intersection between the fourth intercostal space at the sternum and the midpoint between the anterior and posterior aspects of the chest wall. The device 100 may be adhered directly to the patient's skin or indirectly via an intermediate element, such as a docking structure (as disclosed herein), a bandage, and/or an article of clothing.

In those embodiments in which a docking structure is used, the method may optionally include determining whether the device 100 has been properly secured to the docking structure. As disclosed herein, the device 100 and/or docking structure may include one or more complementary sensors for detecting close proximity and/or contact between the device 100 and the docking structure. If the device 100 is properly secured, the device 100 and/or docking structure may optionally provide an indication of attachment to one another. For example, the device 100 and/or docking bracket may include a visual indicator, such as a light, that turns on when proper attachment is achieved. In some embodiments, confirmation of proper attachment and/or lack of attachment may be indicated on the display of the device 100 (e.g., by a symbol indicative of attachment or lack of attachment, by text, etc.). In these and other embodiments, the device 100 and/or docking structure can be configured to output auditory confirmation of the connection.

The device 100 may be powered on prior to attachment to the body (or docking structure) or after, for example via the input device (e.g., a button, switch, etc.). In some cases, coupling of the device 100 to the docking structure automatically powers on the device 100.

Once the device 100 is secured to the patient's body, the user can flush the device 100 to purge air from the flow channel 124 and/or pressure sensor 114 (block 304). In some embodiments, the device 100 may be flushed prior to coupling to the body. In any case, performing a flushing operation can be especially beneficial when utilizing a pressure sensor comprising a fluid-filled transducer (as detailed herein). In some examples, the device 100 prompts the user (e.g., via the display, via an auditory command, via an indication light on the housing 102, etc.) to perform a flushing operation and/or otherwise indicates that the device 100 is ready to be flushed. To flush the device 100, the user may couple a fluid source (e.g., a saline syringe, a saline bag, etc.) to the first port 104 and begin pushing fluid through the device 100 and/or channel 124. In those embodiments of the device 100 that include a built-in reservoir (as disclosed herein), the user may activate a flushing operation by manipulating an actuator (e.g., a button, knob, switch, etc.) on the housing 102 and/or the display. In some embodiments, initiation of a flushing operation (e.g., providing visual or auditory instructions to the care provider) occurs automatically when proper attachment of the device 100 to the docking bracket is detected. In some examples, successful flushing of the device 100 and/or pressure sensor 114 can be automatically detected via one or more sensors and/or algorithms within the device 100, and the device 100 may provide an indication of completion of the flushing operation to the user (e.g., via the display, via an auditory command, via an indication light on the housing 102, etc.). The device 100 may automatically detect completion of the flushing operation (e.g., via one or more flow sensor disposed in the channel 124 and coupled to the processor 118) and/or the device 100 may ask the care provider (e.g., via the display) to provide an input (via one or more input devices, such as a button on the housing 102) to confirm completion of the flushing operation.

As shown at block 306, the method may proceed with obtaining position data of the device 100 via the position sensor 116. In some cases the device 100 automatically proceeds with obtaining position data after the flushing operation has been performed (which as previously mentioned may be confirmed by the care provider and/or detected by the device 100).

For the first and/or reference position measurement(s), the position data is stored in memory 119 and the care provider proceeds with the method. In some implementations, the device 100 instructs the care provider to proceed via instructions displayed on the output device 120 (such as the display or other visual indicators (e.g., a light, a symbol on the display, etc.)) and/or otherwise indicates via an output device that a reference position has been measured and/or that the user can proceed. For subsequent position measurements, the position data may be stored in the memory 119 and processed by the processor 118 to detect potential changes in patient position. Changes in patient position (and thus device position) between blood pressure measurements can affect the accuracy of the measurements, as such positional discrepancies affect the vertical height of the device 100 and thus introduce hydrostatic pressure changes into the measurements. If the new position data varies from the initial position data by more than a predetermined threshold, the device 100 can do one or more of: (a) calibrate subsequent pressure measurements based on the initial or previous reference position and new position data; (b) alert the user that subsequent blood pressure measurements may be inaccurate; and/or (c) instruct the user (e.g., via the display) to reposition the patient (and thus the device 100). The predetermined variance threshold of patient movement may be, for example, within five degrees of the initial position, within 10 degrees of the initial position, within 15 degrees of the initial position, or within 20 degrees of the initial position. As such, the device 100 ensures that successive blood pressure measurements are taken under identical or nearly identical hydrostatic pressure conditions and that any changes in blood pressure measurements are a result of changing clinical conditions of the patient (e.g., cardiac hemodynamics, fluid status, etc.).

In the instances where the device 100 automatically compensates for patient movement, the patient's measured position may be used with the initial or previous reference position to compute the expected offset in pressure. Such auto-calibration avoids the user having to reposition the patient, which is more comfortable for both the patient and user. In some embodiments, the device 100 informs the user that the new position is outside of an acceptable range of the initial or previous position, and the user decides whether to proceed with blood pressure measurements or reposition the patient. In some cases, if the difference between the previous and current positions is too large (e.g., greater than 20 degrees, greater than 30 degrees, greater than 40 degrees, greater than 45 degrees, greater than 50 degrees, greater than 60 degrees, greater than 70 degrees, greater than 80 degrees, or greater than 90 degrees), the device 100 may instruct the user that recalibration is not recommended and that the patient must be moved in order to proceed.

As previously mentioned, the device 100 may provide instructions via the output device(s) for repositioning the patient. In some implementations, the display communicates the approximate number of degrees that the patient is out of position and the directionality of the required adjustment. For instance, if the patient is initially positioned with their torso at 45 degrees relative to the horizontal plane (as shown in FIG. 2A) for the initial or previous measurement and the device 100 measures the patient to be supine during a subsequent position measurement, the device 100 may communicate to the care provider that the patient needs to be moved 45 degrees upwardly. In some examples, the device 100 does not display the amount of displacement but only that the patient should be moved up or down (via text, arrows, etc.). Likewise, in some cases the device 100 does not display explicit directional guidance but instead provides only an approximate number indicating directionality (e.g., “−45°” from initial position indicates that patient should be moved up 45 degrees, and “+45°” indicates the patient should be moved down 45 degrees.)

After getting the patient in the correct position but prior to obtaining a pressure reading, the pressure sensor 114 can be zeroed to atmospheric pressure for calibration. The zeroing procedure may be initiated manually by the user via the input device 121 (e.g., one or more buttons on the housing 102 or display), or automatically when the device 100 is powered on and senses that the pressure sensor 114 is open to air (e.g., disconnected from the patient). The latter scenario allows zeroing to occur without the need for human input, eliminating potential human error. In some embodiments, the device 100 includes a valve operatively coupled to the pressure sensor 114 and configured to be automatically or manually opened and closed to expose the pressure sensor 114 to air. In such embodiments, the zeroing procedure may be performed by pushing a button on the housing 102 or display of the device 100, which causes the valve to open and close. In some embodiments, opening of the valve to air causes the pressure transducer to be exposed to air, while simultaneously closing off exposure of the pressure transducer to the orifice that is exposed to the tubing connected to the peripheral intravenous line.

The method can optionally include detecting whether the pressure sensor 114 is open to air during the zeroing procedure, and alerting the user if the zeroing procedure is being performed under the wrong conditions (e.g., after the device 100 has been fluidly coupled to the patient, rather than before). In some instances, the device 100 may recognize that a false zeroing procedure has been performed by detecting the presence of physiologic signals from a patient blood vessel that are distinct from expected atmospheric pressure readings. For example, blood pressure signals showing pulsatile flow, or that are variable with an expected respiratory rate, may indicate that the pressure sensor 114 is exposed to the patient's blood vessel rather than atmospheric air as intended during a zeroing function. In that case, the device 100 can provide an alert to the user, either via the display, an audible signal, a visual indicator (e.g., a light) on the housing 102, etc. In some instances, the device may compensate for the reference pressure being incorrect when a zeroing calculation has been performed incorrectly.

The method proceeds with connecting the device 100 to the fluid source 106 and the patient and optionally instructing the user to perform a patency check to ensure that the fluid channel and/or cannula is not blocked and/or kinked. In some embodiments, the patency check may comprise an arm squeeze test. For example, the device 100 may prompt the user (e.g., via the output device 120) to gently squeeze the patient's arm while the device 100 obtains pressure measurements. If the flow channel and/or cannula are sufficiently patent, then the pressure measurements should reflect an increase in pressure, or at least an increase in pressure greater than a predetermined threshold (e.g., an increase of at least 5, 10, 15, 20, 25, or 30 mmHg), or at least greater than the typical increase in pressure between systole and diastole (e.g., between the trough and peak in a pressure waveform). If, however, in response to the arm squeeze, the device 100 does not measure an increase in pressure, or does not measure an increase in pressure greater than a predetermined threshold, then the device 100 alerts the user that one of the flow channel and/or cannula are not sufficiently patent. In some cases the user provides an input to the device 100 to start a patency check, which causes the device 100 to look for a predetermined increase in pressure for a predetermined amount of time following the initiation of the patency check. If the device 100 does not detect the expected pressure increase, the device 100 may communicate to the user (e.g., via the display or other indicator) that the flow channel and/or cannula are not sufficiently patent. If the device 100 detects the expected pressure increase during the time period of the patency test, the device 100 alerts the user that patency is confirmed and the device is ready to measure.

The predetermined threshold for the patency check can be based on patient averages, or may be tailored to the patient. For example, the device 100 can take use previously-obtained maximum and minimum pressure values for a particular patient to determine an appropriate patency check threshold for that patient, or use a look up table stored in the memory.

In addition to or instead of evaluating patency by detecting an expected increase in pressure, the device 100 may utilize other parameters to detect an obstruction and/or kink. For example, the device 100 may be configured to detect flatlining, decreases in pressure greater than a predetermined threshold, loss of pulsatility, loss of the entire waveform, etc.

As shown at block 308, the device 100 can begin taking blood pressure measurements, either automatically or by initiation from the user (e.g., via a button on the display, a button on the housing 102, etc.) While the blood pressure monitoring is underway, the device 100 may continue to monitor the position of the patient ensure that the blood pressure measurements are accurate. If changes in patient position greater than a predetermined threshold are detected (and the device 100 is not compensating for the change in patient position), the device 100 can instruct the user to reposition the patient, as previously described herein.

While the device 100 is measuring blood pressure, the device 100 may display a blood pressure value on the display. As but one example, in some embodiments the cannula may be positioned in the antecubital vein (e.g., in the arm or leg) such that the device 100 can directly measure PVP and display a mean PVP value on the display. Blood pressure measurements may be performed for any suitable period of time (e.g., at least 10 seconds, at least 20 seconds, at least 30 seconds, at least one minute, at least two minutes, etc.) and stored in the memory of the device and/or transmitted via a wireless transmitter in the device to a remote signal processing unit on a mobile or stationary device (e.g., phone, tablet, computer).

In some embodiments, the device 100 may assess the respiratory cycle of the patient using the position sensor 116 data to detect timing of expiration and inspiration. Since venous pressure measurements may vary with inspiration and expiration (e.g., decrease with expiration), a defined timing point of the respiratory cycle (e.g., end-of-expiration) is selected for measurement of venous pressure to improve consistency when comparing measurements.

FIGS. 4A and 4B are top and perspective views, respectively, of a device 400 for measuring blood pressure configured in accordance with several embodiments of the present technology. The device 400 can comprise a housing 402, a first port 404, and a second port 410, similar to the housing, first port, and second port of device 100. The device 400 includes an output device 420 comprising a display screen and an input device 421 comprising a button, positioned adjacent the display. The button may be configured to power the device on and off, as well as confirm user steps instructed by the device 100, such as beginning a zeroing operation, beginning a patency check (as discussed herein), taking a blood pressure measurement, etc. The device 400 may further include a pressure sensor, a position sensor, a processor, memory, a power source, and external markings 440, all carried by the housing 402 and similar to the pressure sensor 114, position sensor 116, processor 118, memory 119, and power source 122 described above with reference to FIG. 1. The device 400 is shown in FIGS. 4A and 4B displaying a blood pressure value.

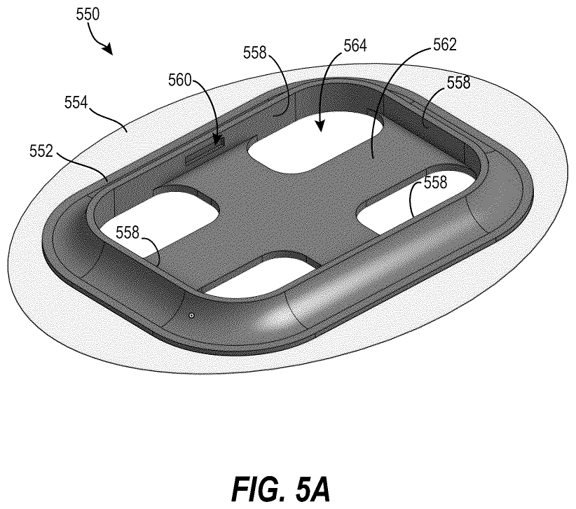

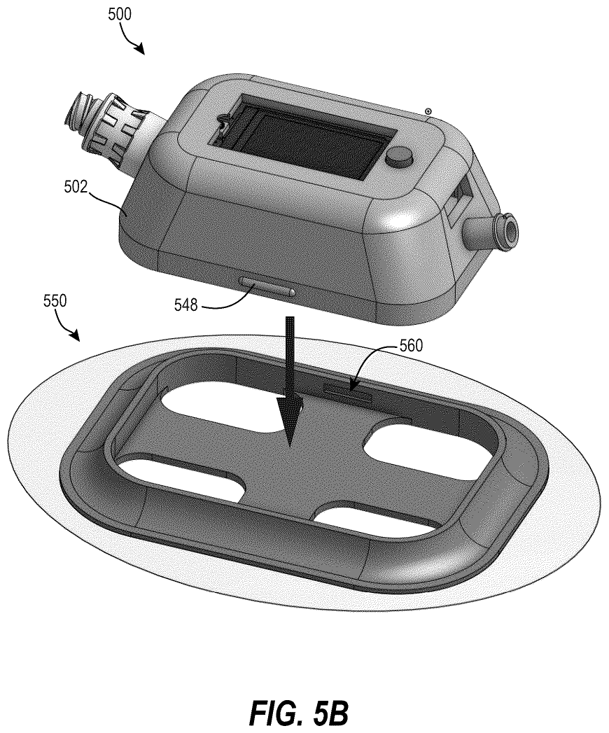

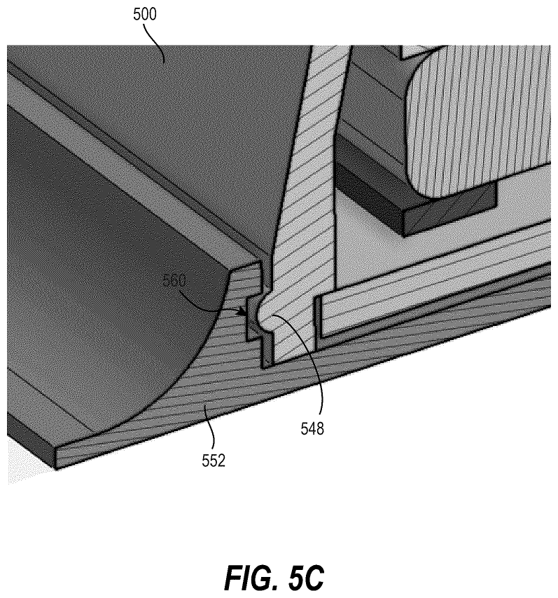

FIG. 5A shows a docking structure 550 for use with the blood pressure measuring devices of the present technology, and FIG. 5B shows the docking structure 550 prior to coupling with an example device 500. The docking structure 550 is configured to be secured to the patient's body (e.g., at the chest wall and/or phlebostatic axis) and detachably couple to a device for measuring blood pressure (such as any of the devices 100, 400, 600, 700, etc.) disclosed herein. The docking structure 550 advantageously allows the device to be repeatedly removed and re-installed at the same location on the patient's body, thereby preventing inaccuracy and imprecision of pressure monitoring due to changes in device position on the body. The docking structure 550 maintains the initial measurement position without the device needing to be present on the patient for long periods of time.

As shown in FIGS. 5A and 5B, the docking structure 550 includes a base 552 having a bottom surface coupled to an adhesive patch 554, which is placed on the skin of a patient. The adhesive patch 554 may be configured to be adhered to the patient's skin for up to several days (up to 14 days). The docking structure 550 may further include sidewalls 558 extending upwardly from the base 552 and configured to surround all of a portion of a bottom sidewall of the housing 102 when the device 100 is coupled to the base 552. A bottom wall 562 of the docking structure may have one or more openings 564 (as shown), or may be solid.

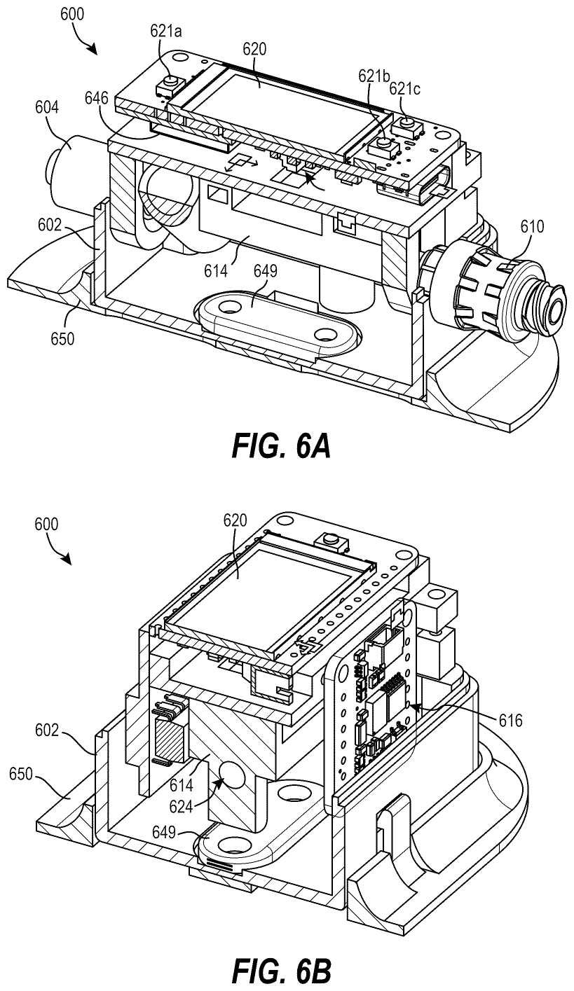

The docking structure 550 may further include one or more coupling elements and/or features configured to detachably couple to one or more complementary coupling elements and/or features on the housing 102. For example, as shown in FIGS. 5A and 5B, any of the device herein can include a protrusion 548 extending outwardly from a bottom aspect of one, some, or all four sidewalls of the housing 502. In FIGS. 5A and 5B, only two opposing sidewalls of the housing 502 include the protrusion 548. The docking structure 550 can include a recess 560 extending inwardly into a sidewall 558 of the device and configured to mate with the protrusion 548 on the device. FIG. 5C is an enlarged cross-sectional view showing the protrusion 548 received within the recess 560. In some implementations, the device includes a recess in the housing 502 and the docking structure includes a protrusion. Other forms of coupling are possible. For example, in some embodiments the docking structure includes a deformable ledge configured to engage a surface of the housing. The device can be pushed downwardly into the docking structure such that the ledge engages the surface (for example, as shown in FIGS. 6A and 6B) and prevents relative movement along a vertical axis between the device and the docking structure. In any case, the device may be coupled to the docking structure by snapping and/or pushing the device downwardly into the recess defined by the sidewalls 558 of the docking structure. In some implementations, the device may be configured to slide laterally into the docking structure for securement.

In some embodiments, the device 100 and/or housing 102 may include one or more proximity sensors to detect the presence of the docking structure 550 and confirm attachment. For example, the device 100 can include one or more Hall sensors carried by the housing 102 and configured to provide binary or analog output in the presence of a magnet 556 disposed on the docking structure 550. In some embodiments, the magnet may be magnetized in a way that allows for various orientations of the magnet to be sensed by the electronics in the housing 102 for identification purposes. In some embodiments, connection and/or identification may be sensed by optical sensors, by mechanical switches, by magnetic sensing, by inductive sensing, by RFID, etc. In some examples, the device 100 may not be permitted to obtain a blood pressure measurement unless it is sensed to be within the docking structure 550 placed at the chest, in order to ensure accurate measurements. In some implementations, the proximity sensor(s) comprises a magnetic rotary encoder that senses not only the presence of a magnetic field but also its rotation. This sensor may be used to produce a unique identifier that is used in the device logic. Additional means of sensing attachment to the docking structure may be via mechanical switches, electrical contacts, RFID sensors, and/or optical sensors.

In some embodiments, the docking structure 550 may have features that allow for easy marking and/or alignment of the docking structure 550 during initial placement or when removal and replacement are necessary. In some embodiments, the docking structure 550 acts as a self-contained sensing unit with associated electronics. In some embodiments, the adhesive could contain conductive material to allow for additional various sensing modalities, such as ECG or ultrasound. The adhesive could also contain electrical traces that function as sensors or antennas for usages such as but not limited to communication, charging, and sensing.

The present technology may optionally include a temporary marking configured to be applied to the patient's chest wall at the desired device location, instead of or in addition to use of the docking structure. After securing the device to the patient for the first time, or at any time when the care provider has determined a desirable position for securement, the care provider may mark the patient's chest (e.g., with a non-toxic ink) at various points around all or a portion of the perimeter of the device. In some cases, the care provider may determine the location prior to attachment of the device. The care provider may mark the desired location by applying a tattoo having regions corresponding to all or a portion of the perimeter of the device, or simply mark the patient's skin with a non-toxic ink. The care provider can then align the device with the tattoo or markings for future measurements.

FIGS. 6A and 6B are cross-sectional side and axial views, respectively, of a device 600 for measuring blood pressure configured in accordance with several embodiments of the present technology. In FIGS. 6A and 6B, the device 600 is shown secured to a docking structure (which is also shown in cross-section). The device 600 can comprise a housing 602, a first port 604, and a second port 610, similar to the housing, first port, and second port of device 100. The device 600 includes an output device 620 comprising a display screen and a plurality of input devices 621 (labeled individually as 621a, 621b, and 621c), each comprising a button and positioned adjacent the display at a top surface of the device 600. In other embodiments, the display and buttons may be disposed at different surfaces of the housing 602 (e.g., the display and at least one of the buttons may be on the top surface while another of the buttons may be on a side surface, the display may be on the top surface while all of the buttons are on a side surface, the display may be on a side surface and some or all of the buttons are on the top surface, etc.). One of the buttons 621 may be configured to power the device on and off while another of the buttons 621 is configured to confirm user steps instructed by the device 600, such as beginning a zeroing operation, beginning a patency check (as discussed herein), taking a blood pressure measurement, etc. The device 600 may further include a pressure sensor 614, a channel 624, a position sensor 616, a wireless transmitter 646, a processor, memory, and a power source, all carried by the housing 602 and similar to the pressure sensor 114, channel 124, position sensor 116, wireless transmitter, processor 118, memory 119, and power source 122 described above with reference to FIG. 1.

In some embodiments, and as shown in FIGS. 6A and 6B, the device 600 can include a proximity sensor 649 at the bottom interior surface of the housing 602 that is configured to detect the presence of the docking structure, for example via interaction with a complementary device on the docking structure (as described herein). The sensor 649 can be coupled to the processor and/or an output device so that the device 600 can communicate to a user (via an output device) that the device 600 is secured to the docking structure 650.

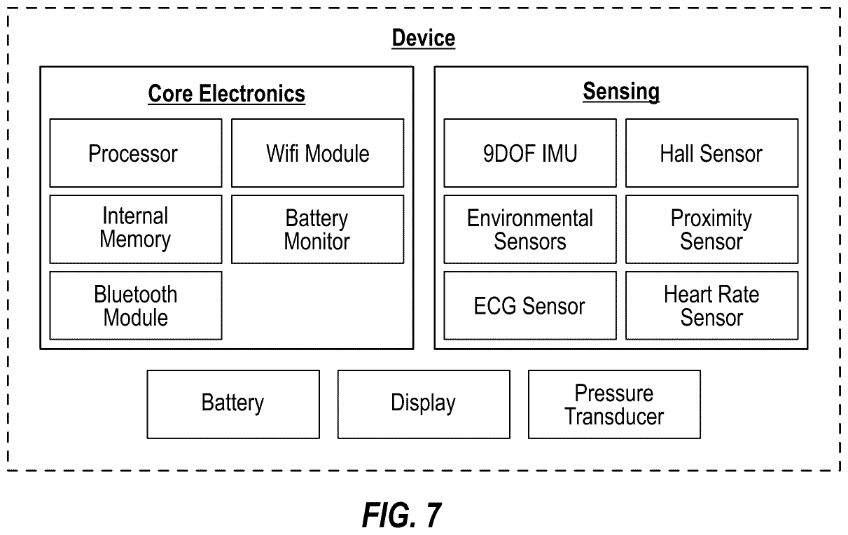

FIG. 7 is a block diagram of a device for measuring blood pressure configured in accordance with several embodiments of the present technology. As shown, the device can include a processor, a WiFi module, internal memory, a battery monitor, a Bluetooth module, a battery, a display, and a pressure transducer. The device may further include a 9 dof IMU, a Hall sensor, one or more environmental sensors, a proximity sensor, an ECG sensor, and a heart rate sensor.

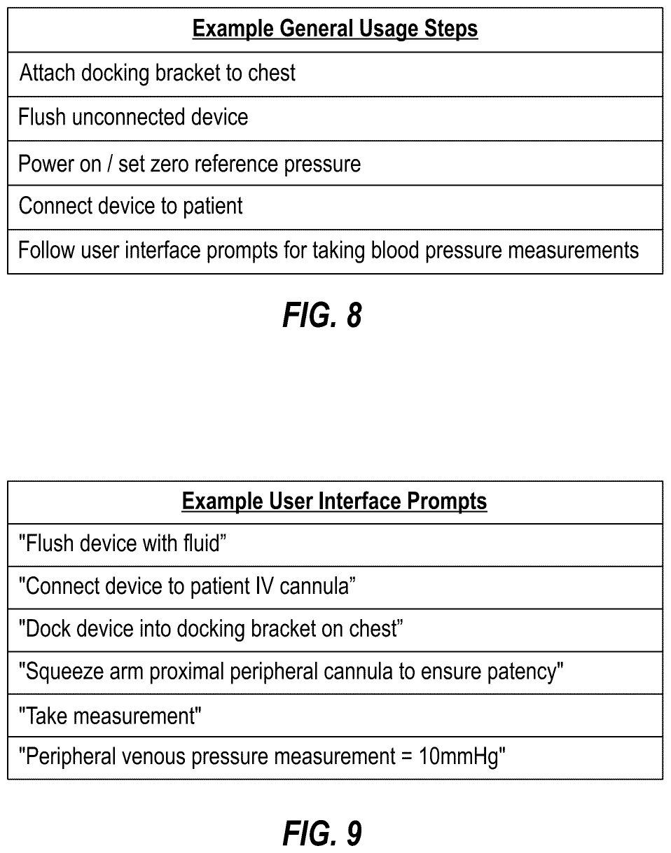

FIG. 8 shows an example method for using the devices of the present technology to measure blood pressure. For example, some methods of use comprise securing the device to the patient's body (as disclosed here), either directly or via the docking structure, and before connecting the device to the catheter or fluid source (if applicable), performing a flushing operation as described herein. The method further includes powering on the device (via one or more inputs) and zeroing the device, which may occur automatically upon powering on the device or may occur as a separate step (as detailed herein). The second port of the device may then be fluid coupled to a catheter with a distal end positioned in the patient's blood vessel. Finally, the method includes following prompts on the device output (such as a display screen) to take the blood pressure measurement. The device may also include user prompts when performing a zeroing operation and/or to reposition the device.

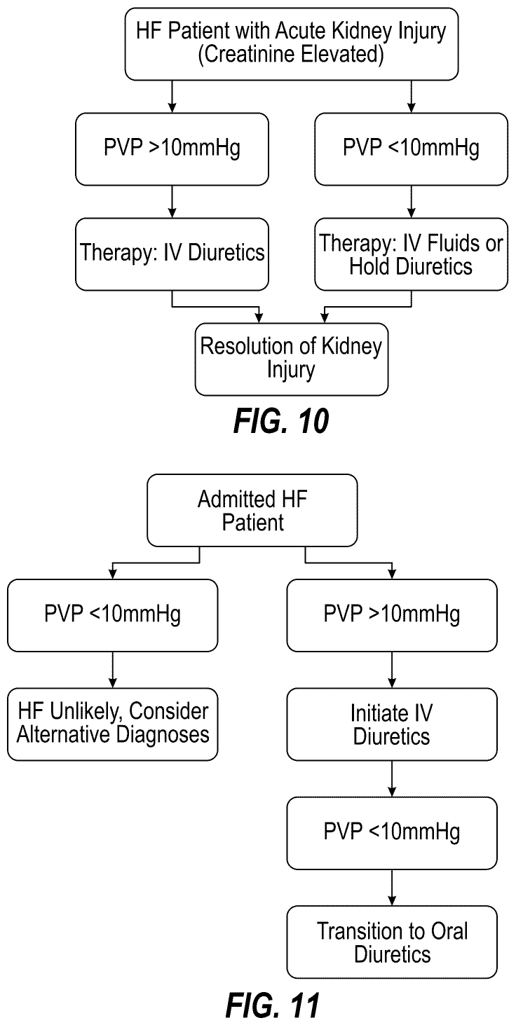

FIG. 9 is a table showing example output prompts of a device configured in accordance with the present technology. Example prompts include “Flush device with fluid,” or other text conveying instructions to the user for performing a flushing operation; “Connect device to patient IV cannula,” or other text conveying instructions to the user for connecting another element to the first and/or second port; “Dock device into docking bracket on chest,” or other text conveying instructions for securing the device and/or the device and docking structure assembly to the patient's body and/or for securing the device to the docking structure (whether on the patient's body already or not); “Squeeze arm proximal peripheral cannula to ensure patency” or “Perform squeeze test” or other text conveying instructions for performing a patency check; “Take measurement” or “Press button to take measurement” or “Ready for measurement” or other text conveying instructions to begin taking a blood pressure measurement; and/or “Peripheral venous pressure measurement=10 mmHg” or just “_____ mmHg” or other text to convey a blood pressure measurement value. It will be appreciated that the device may display and/or output other user guidance as disclosed herein, including positioning guidance, such as “Reposition device” or “Sit patient up” or “Lay patient back,” or simply arrows pointing up or down in the direction of the desired repositioning.

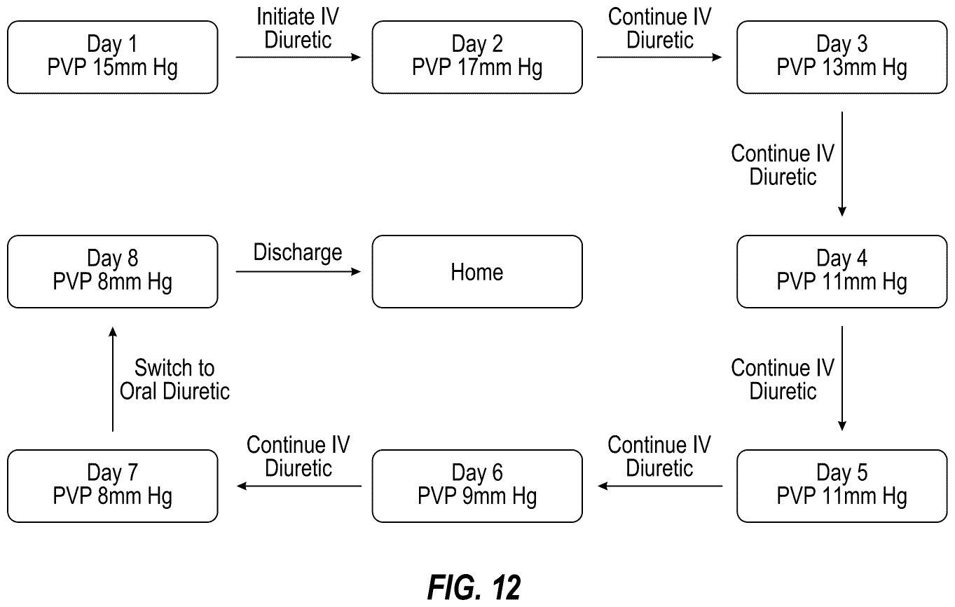

FIGS. 10, 11, and 12 show clinical applications of the blood pressure measuring devices of the present technology. A mean PVP and/or its trend may be used in conjunction with other clinical data (e.g., creatinine, renal function) to make clinical decisions. For example, a patient may be admitted to the hospital with heart failure and acute kidney injury, a condition known as cardiorenal syndrome. In these patients, diuretic decisions can be difficult to make due to how challenging it can be to verify whether patients are dry or congested, as acute kidney injury is often traditionally attributed to a volume depleted state but may also be caused by congestive states such as decompensated heart failure. As such, the devices of the present technology can be use to measure blood pressure and, in conjunction with the patient's symptoms, devise an appropriate treatment plan.

FIG. 10 is a flow diagram illustrating a treatment path that incorporates PVP in conjunction with creatinine and renal function information to make clinical decisions for a patient, in accordance with embodiments of the present technology. The treatment path begins with identification of a patient diagnosed with cardiorenal syndrome and who demonstrates elevated creatinine suggestive of kidney injury. A PVP measurement is taken (via the devices of the present technology) and evaluated before the treatment plan diverges to provide the corresponding suitable treatment to the patient based on their condition. A high PVP measurement (e.g., above 10 mmHg) in conjunction with elevated creatinine results in the patient receiving IV diuretics, which helps with decongestion and is an appropriate strategy to treat both heart failure and kidney injury. After sufficient therapy has been provided to the patient based on the severity of their condition, resolution of kidney injury is achieved. Conversely, a low PVP measurement (e.g., below 10 mmHg) in conjunction with elevated creatinine indicates a volume depleted state of the patient and the corresponding treatment is provided to the patient. Diuretics may be held and/or fluid replacement therapy is given to the patient. Following sufficient therapy to manage the patient's condition, resolution of kidney injury is achieved.

In some embodiments, mean PVP may be checked daily (via the devices of the present technology) and monitored for trends to help guide diuretic decisions in patients admitted with decompensated heart failure. For example, a patient with chronic heart failure may be admitted to the hospital with symptoms suggestive of heart failure such as shortness of breath. PVP measurements may be used to inform and guide decisions on treatment with diuretics or to determine if heart failure is the correct diagnosis for that particular patient.

FIG. 11 is a flow diagram illustrating a treatment path that incorporates repeated PVP measurements (using the devices of the present technology) to guide clinical decisions for a patient, in accordance with embodiments of the present technology. The treatment path begins with a chronic heart failure patient being admitted to the hospital. A first PVP measurement is taken for the patient and evaluated before the treatment plan diverges to provide the corresponding suitable treatment to the patient based on their condition. If the patient's PVP measurement is low (e.g., below 10 mmHg), the measurement is taken into consideration along with the patient's symptoms prior to determining next steps for treatment. Here, it is determined that heart failure is unlikely and that alternative etiologies may need to be considered for the patient. Conversely, if the patient's PVP is high (e.g., above 10 mmHg), the patient is evaluated for other symptoms suggestive of heart failure and that may indicate that decompensated heart failure is occurring. Next, IV diuretics are initiated for the patient. After IV diuretics have been administered to the patient for a suitable amount of time, an additional PVP measurement is taken to assess the status of the patient. If the PVP measurement is low (e.g., below 10 mmHg), then the treatment provided to the patient is transitioned to oral diuretics.

In some embodiments, mean PVP can be measured with the devices disclosed herein intermittently either once or twice daily over a longer period of time to inform clinical decisions on whether to continue administrating IV diuretics and/or when to transition to oral diuretics for a patient demonstrating elevated PVP. If PVP is elevated, the clinician may initiate IV diuretics and continue IV diuretics while the measured PVP remains elevated relative to a target PVP threshold (e.g., 8 mmHg). After the target PVP (e.g., at and/or below 8 mmHg) has been achieved, IV diuretics may be transitioned to oral diuretics and the patient may be discharged home. This may help to avoid cases in which patients are discharged home with residual congestion as patient symptoms tend to improve faster than PVP normalizes. Discharging patients home prior to PVP normalization may increase the risk of readmission due to residual congestion. In some embodiments, PVP monitoring may be performed using a PVP monitoring patch intermittently or twice daily in non-critical care settings (e.g., emergency department, general medicine, cardiology ward) to help guide diuretic decisions. For example, PVP measurements may be used to inform decisions to increase, decrease, or continue IV diuretics, switch to oral diuretics, provide intravenous fluid therapy, and/or discharge a patient home from the hospital.

FIG. 12 is a flow diagram illustrating an example treatment path for a chronic heart failure patient demonstrating elevated PVP, in accordance with embodiments of the present technology. The example treatment path begins with measuring the PVP of the patient (via the devices of the present technology) to be 15 mmHg on Day 1. Since PVP is elevated (e.g., above 8 mmHg), the clinician can initiate administering IV diuretic to the patient. Another PVP measurement of the patient is taken on Day 2. The measured PVP is 17 mmHg, which is still elevated and the clinician can continue administering IV diuretic to the patient. A PVP measurement of 13 mmHg is taken on Day 3, which is still elevated and the clinician can continue administering IV diuretic to the patient. Another PVP measurement of 11 mmHg is taken on Day 4, which is still elevated and the clinician can continue administering IV diuretic to the patient. Another PVP measurement of 11 mmHg is taken on Day 5, which is still elevated and the clinician can continue administering IV diuretic to the patient. Another PVP measurement of 9 mmHg is taken on Day 6, which is still elevated and the clinician can continue administering IV diuretic to the patient. Another PVP measurement of 8 mmHg is taken on Day 7, which indicates that the target PVP has been reached and the clinician can transition the patient to oral diuretic. Another PVP measurement of 8 mmHg is taken on Day 8, which indicates that the target PVP has still been maintained and the patient can be discharged.

### II. Other

Any of the devices disclosed herein may include components configured to be completely disposable. In this example, this allows for a fully sterile monitoring device that is intended for a single patient but can be used intermittently for a defined period of time such as a single hospitalization. When the device is not being used or is not connected to the patient's cannula within a blood vessel such as a peripheral intravenous cannula, the patient-facing end of the system may be capped with a luer-lock cap system to prevent device contamination. Prior to re-use of the device within a single hospitalization on a single patient, the patient-facing end may be wiped with a sterile alcohol swab again to prevent the risk of infection. In some examples, the display system may include reminders to users such as nurses for the device to maintain device hygiene.

For a reusable embodiment, the components may be split into two or more housings to allow for one portion to be reused and the other to be disposable. One embodiment of this might be a disposable housing that contains the pressure sensor and electrical contacts that allows for mating of another reusable housing(s) that contain the measurement electronics and user interface/feedback elements. Another embodiment could couple a disposable pressure measurement unit with wireless communication capability to allow for the measurements to be read and recorded on a variety of separate reusable devices.

In some examples, the device 100 may be delivered in packaging as a pre-assembled device including the housing and adhesive docking station connected together. In some examples, after sterile use with a single patient is complete (e.g., at the end of an admission), patient-facing components (for example adhesive or fluid-filled pressure sensor) may be easily removed and discarded, while the remaining electronic components such as LCD display and/or IMU, and/or circuit boards etc., may be kept for re-use. In some examples, the remaining electronic components may be collected by the manufacturer for re-use in manufacturing. In some examples, the entire PVP monitoring system including the patient-facing components may be collected by the manufacturer, and the non-patient-facing components may be stripped and recycled in the manufacturing center for reuse.

Any of the devices disclosed herein can include components and functions to obtain PVP to meet the following needs: (1) obtain PVP measurements in areas in which a monitor is not readily available (for example hospital ward); (2) obtain PVP measurements daily that are both accurate and precise, accounting for potential errors that may change measured PVP independent of PVP alone, such as patient position; (3) provide guidance to users that may be unfamiliar with PVP or pressure sensing technologies in order to deploy and obtain signals using a Device 100; (4) provide a fully portable solution that is plug-and-play out of the box, not requiring existing capital equipment or infrastructure to use; (5) combine information from multiple sensors to provide information around the cardiac status of patient; (6) measure PVP signals to obtain an estimate of central venous pressure or other intracardiac pressures such as pulmonary artery pressures, left atrial pressures, left ventricular pressures; and (7) measure PVP signals to manage patients with volume overload states such as heart failure or volume depletion states such as acute kidney injury.

### CONCLUSION

Although many of the embodiments are described above with respect to systems, devices, and methods for measuring PVP, the technology is applicable to other applications and/or other approaches, such as measurement of other blood pressures, such as any venous pressure (including central venous pressure) and any arterial pressure. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1A-10.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A device for measuring intravascular pressure, the device comprising:
a housing configured to be secured to a patient's body;
an output device carried by the housing;
a pressure sensor configured to obtain pressure data;
a position sensor configured to obtain position data;
at least one processor in communication with the pressure sensor and the position sensor;
at least one tangible, non-transitory computer-readable medium; and
program instructions stored on the at least one tangible, non-transitory computer-readable medium that, when executed by the at least one processor, cause the device to perform functions comprising:
based on position data received from the position sensor, indicate to a user that the device and/or patient is in position for a pressure measurement or that the device and/or patient should be repositioned, and
based on pressure data received from the pressure sensor, determine a blood pressure of the patient and cause the output device to indicate to the user the blood pressure of the patient.

2. The device of claim 1, wherein the position sensor is configured to measure the position of the housing with three degrees of freedom.

3. The device of claim 1, wherein the position sensor is configured to measure the position of the housing with six degrees of freedom.

4. The device of claim 1, wherein the position sensor is configured to measure the position of the housing with nine degrees of freedom.

5. The device of claim 1, wherein the position sensor comprises an accelerometer.

6. The device of claim 1, wherein the position sensor comprises a gyroscope.

7. The device of claim 1, wherein the position sensor comprises a magnetometer.

8. The device of claim 1, wherein the position sensor comprises an inertial measurement unit.

9. The device of claim 1, wherein the output device includes a display carried by the housing, and wherein the processor is coupled to display and configured to output the determined blood pressure on the display.

10. The device of claim 9, wherein the determined blood pressure is output as a number.

11. The device of claim 9, wherein the determined blood pressure is not displayed as a waveform.

12. The device of claim 9, wherein the processor is configured to output instructions for a user on the display.

13. The device of claim 1, wherein the blood pressure comprises mean venous pressure.

14. The device of claim 1, wherein the blood pressure comprises peripheral venous pressure PVP.

15. The device of claim 1, wherein the processor is disposed within the housing.

16. The device of claim 1, further comprising an input device carried by the housing and coupled to the processor.

17. A device for measuring intravascular pressure, the device comprising:
a housing configured to be secured to a patient's body;
an output device carried by the housing;
a pressure sensor configured to obtain pressure data;
a position sensor configured to obtain position data;
at least one processor in communication with the pressure sensor and the position sensor;
at least one tangible, non-transitory computer-readable medium; and
program instructions stored on the at least one tangible, non-transitory computer-readable medium that, when executed by the at least one processor, cause the device to perform functions comprising:
determine a blood pressure of the patient based on the pressure data and the position data; and
output the blood pressure via the output device.

18. The device of claim 17, wherein the position sensor is configured to measure the position of the housing with three degrees of freedom.

19. The device of claim 17, wherein the position sensor is configured to measure the position of the housing with six degrees of freedom.

20. The device of claim 17, wherein the position sensor is configured to measure the position of the housing with nine degrees of freedom.

21. The device of claim 17, wherein the position sensor comprises an accelerometer.

22. The device of claim 17, wherein the position sensor comprises a gyroscope.

23. The device of claim 17, wherein the position sensor comprises a magnetometer.

24. The device of claim 17, wherein the position sensor comprises an inertial measurement unit.

25. The device of claim 17, wherein the output device comprises a display carried by the housing, and wherein the processor is coupled to display and configured to output the determined blood pressure on the display.

26. The device of claim 25, wherein the determined blood pressure is output as a number.

27. The device of claim 25, wherein the determined blood pressure is not displayed as a waveform.

28. The device of claim 25, wherein the processor is configured to output instructions for a user on the display.

29. The device of claim 17, wherein the blood pressure comprises mean venous pressure.

30. The device of claim 17, wherein the blood pressure comprises peripheral venous pressure PVP.

31. The device of claim 17, wherein the processor is disposed within the housing.

32. The device of claim 17, further comprising an input device carried by the housing and coupled to the processor.
